(19) United States
(12) Patent Application Publication
(10) Pub. No.: US 2016/0081620 A1
(43) Pub. Date: Mar. 24, 2016

(54) METHOD AND APPARATUS FOR HEALTH CARE

(71) Applicant: SAMSUNG ELECTRONICS CO., LTD., Suwon-si (KR)

(72) Inventors: Rangavittal NARAYANAN, Bangalore (IN); Vijay Narayan TIWARI, Bangalore (IN); Saswata SAHOO, Bangalore (IN); Mithun Manjnath NAYAK, Bangalore (IN); Shankar M. VENKATESAN, Bangalore (IN); Aloknath DE, Bangalore (IN); Vivek JILLA, Bangalore (IN); Choong-hyun LEE, Suwon-si (KR); Subramanian RAMAKRISHNAN, Bangalore (IN); Ramachandran NARASIMHAMURTHY, Bangalore (IN); Avinash PRASAD, Bangalore (IN)

(73) Assignee: SAMSUNG ELECTRONICS CO., LTD., Suwon-si (KR)

(21) Appl. No.: 14/858,833

(22) Filed: Sep. 18, 2015

(30) Foreign Application Priority Data
Sep. 19, 2014 (IN) 4582/CHE/2014
Jul. 24, 2015 (IN) 3821/CHE/2015
Sep. 14, 2015 (KR) 10-2015-0129777

(51) Int. Cl. A61B 5/00; A63B 24/00; A61B 5/0205

(52) U.S. Cl. CPC A61B 5/4866; A61B 5/0205; A61B 5/7278; A61B 5/7275; A61B 5/7267; A63B 24/0062; A63B 24/0075; A61B 5/024

(57) ABSTRACT

An apparatus includes: a receiving unit for receiving a sensor signal for a body of a user from a wearable apparatus; a controller for classifying a physical activity of the user as one of a plurality of predefined activity models based on the received sensor signal, and generating prediction information about the body of the user based on a result of the classifying and profile information about the user; and an output device for outputting health care information to the user based on the prediction information.

### CROSS-REFERENCE TO RELATED APPLICATIONS

This application claims the benefit of Indian Patent Application No. 4582/CHE/2014 filed on Sep. 19, 2014 and Indian Patent Application No. 3821/CHE/2015 filed on Jul. 24, 2015, in the Indian Patent Office, and Korean Patent Application No. 10-2015-0129777 filed on Sep. 14, 2015, in the Korean Intellectual Property Office, the disclosures of which are incorporated herein in their entirety by reference.

### BACKGROUND

1. Field

The present disclosure relates to methods and apparatuses for health care, and more particularly, to methods and apparatuses for health care specialized for a user by using a wearable sensor signal.

2. Description of the Related Art

Various automated apparatuses exist for promoting and maintaining health and wellness. Some of these apparatuses are directed to healthcare data management used by professors at public health colleges, heath care professionals, patients, or all of them. Some of existing data management apparatuses may monitor and record vital statistics. However, in the existing apparatuses, it may be limited to detect motions or activities of a user and suggest health tips and calorie intake. Additionally, a model specialized for a user (that is, a model that may be applied only to a specific user) may be needed to suggest or advise the user based on the detected motions and activities of the user.

### SUMMARY

Provided are methods and apparatuses for health care using a wearable sensor signal.

Provided is a non-transitory computer-readable recording storage medium having stored thereon a computer program which, when executed by a computer, performs the method

Additional aspects will be set forth in part in the description which follows and, in part, will be apparent from the description, or may be learned by practice of the presented exemplary embodiments.

Provided are a method and apparatus for estimating daily energy expenditure during different activities through accurate activity recognition and expended calorie estimation. Further, the method and apparatus may provide personalized models for predicting trends in weight change management. The method and apparatus may provide a signal processing based on an approach, so as to identify and profile an Individual's physical activity seamlessly by using sensor data and map expended calories to an activity corresponding to the expended calorie.

Intensity levels of an activity may be determined by using a fundamental physical parameter in the form of heart rate data, which has not been considered hitherto by other modeling engines, and a physical activity may be associated with specific cardiac rate zones.

Personalization of a model may be performed by training a model by using particular individual sensor data. A health and fitness-related data set for every individual is clinically known to be unique, and hence, development of such a model may lead to personalization. Periodical re-training of the apparatus may lead to adaptive modeling.

The method and apparatus may accurately estimate a nature of a correlation between instantaneous signals from a gyroscope, an accelerometer, and a magnetometer which are generated by movement of hands, legs, or a body while various physical activities are performed (including but not limited to standing, sitting, walking, jogging, sprinting, hiking, climbing/descending stairs, cycling), and use this correlation to develop a highly accurate determination mechanism for various human physical activities.

Different types of sensors (such as a gyro providing features for climbing stairs and an accelerometer providing shock data) situated in a cellular phone held in the same location/position of the body (such as a hand or a pocket) may actually output signals whose phase correlation is highly accurate in classifying the physical activity. Since a magnetometer is known as providing stable directional data, this data can be used to precisely characterize a drift in constants in integrating a distance from an accelerometer. This helps to identify a drastic change in a direction such as turning.

A shape of an accelerometer signal as captured in various transforms (a Fourier boundary descriptor) for each physical activity may improve an accuracy of classification of physical activities that are very close in a feature space.

An accelerometer signal from a specific physical activity which has been studied for a long period of time may provide valuable information about a user. The valuable information may include, for example, (a) an optimal personalized design for walking outfits such as shoes and (b) an impact of terrains on a stride pattern. The method and apparatus may map seamless measurement and tracking of calories (expended over a certain period of time) to a corresponding physical activity, a day-to-day regular activity, and a controlled physical exercise (such as gym workouts).

The method and apparatus may utilize personalized calorie expenditure and endurance data to build models specialized to an individual. Since a model built by training the apparatus with the individual's sensor data is adaptive, the model is subject to change for a certain period of time. Models may be used at various points of time to check one's fitness condition. Robustness of a model depends on an ability of the model to easily pick out a certain changed response of an individual towards an activity or calories expended away from normal, and this may actually result in a deviation of model's central value to a certain degree over a period of time. As an example, since a person's endurance may be improved with a regular exercise, even if the person takes more intensive exercise in a same span of time, the personalized model may be changed adaptively to the improved endurance, and thus, applied to the person.

Intensity level of an activity may be considered as a parameter of a modeling prediction model according to an exemplary embodiment. The Intensity level is estimated by measuring heart rate data during physical activity. The heart rate may be categorized into various cardiac zones.

According to an aspect of an exemplary embodiment, a method and apparatus includes: a receiving unit for receiving a sensor signal for a body of a user from a wearable apparatus; a controller for classifying a physical activity of the user as one of a plurality of predefined activity models based on the received sensor signal, and generating prediction information about the body of the user based on a result of the classifying and profile information about the user; and an output device for outputting health care information to the user based on the prediction information.

The predefined activity models may include at least one selected from the group consisting of a cardio activity, a non-cardio activity, standing, sitting, walking, climbing/descending stairs, hiking, jogging, sprinting, cycling, a treadmill exercise, and driving.

The prediction information may include information about differently predicted calories to be expended to perform the physical activity of the body, according to the activity model obtained by the classifying.

The controller may classify the physical activity of the user as either a cardio activity model or a non-cardio activity model by analyzing the received sensor signal, if the physical activity is classified as the cardio activity model, the prediction information may include information about calories to be expended for the physical activity, the information predicted by performing regression analysis based on heart rate data, and if the activity is classified as the non-cardio activity model, the prediction information may include information about calories to be expended for the physical activity, the information predicted with reference to a calorie chart that shows a relation between the physical activity and calorie expenditure.

The receiving unit may receive sensor signals with respect to the body of the user from a plurality of sensor, and the controller may classify the physical activity of the user as one of the plurality of predefined activity models by using a correlation between the received sensor signals.

The controller may determine a prediction model for generating prediction information about endurance expected in a future of the user, by performing regression analysis based on the received sensor signal.

The prediction information may include information about calories to be expended to perform the physical activity, according to the activity model obtained by the classifying, and the prediction model may be a numerical formula model including at least one variable selected from the group consisting of workout intensity and workout duration and a coefficient determined by least square estimation based on the information about the calories.

The receiving unit may receive heart rate data of the user from the wearable apparatus, and the controller may determine a current endurance level based on the received heart rate data and determine a workout plan for achieving a target endurance level based on the prediction model, and the output device may output to the user health care information that includes the determined workout plan.

The prediction information may include information about a future body weight which is predicted by performing regression analysis by applying regressive integrated moving average (ARIMA) modeling to the received sensor signal.

The sensor signal may be a signal obtained by using at least one wearable sensor selected from the group consisting of a pedometer, a gyroscope, an accelerometer, a heart-rate monitor (HRM), a weight scale, and a barometer.

The apparatus may further include an input device for receiving an input of the profile information from the user, wherein the profile information includes information about at least one selected from the group consisting of a gender, an age, a height, a body weight, and a body mass index (BMI) of the user.

The controller may determine a prediction model for generating the prediction information by performing regression analysis based on the received sensor signal, and the prediction model may be seamlessly recalibrated based on at least one selected from the group consisting of a change in the profile information about the user and a change in endurance of the user.

The output device may display to the user at least one selected from the group consisting of an amount of expended calories, endurance, an recommended amount of food intake, an amount of necessary calorie intake, nutrients or ingredients of consumed food, and a weight change.

According to an aspect of another exemplary embodiment, an apparatus includes a controller for creating an endurance model for predicting endurance of a user by obtaining physical data of the user and user profile information, identifies one or more parameters that affect fitness of the user, and creating a fitness plan for the user based on the identified parameters.

The physical data of the user may include workout data and heart rate data of the user.

The controller may measure the physical data of the user, measure calories consumed and calories expended by the user and create a prediction model, generates an endurance score of the user, and generate health care information of the user for a certain period of time based on the prediction model.

According to an aspect of another exemplary embodiment, a method includes: receiving a sensor signal for a body of a user from a wearable apparatus; classifying a physical activity of the user as one of a plurality of predefined activity models based on the received sensor signal, and generating prediction information about the body of the user based on a result of the classifying and profile information about the user; and outputting health care information to the user based on the prediction information.

The predefined activity models may include at least one selected from the group consisting of a cardio activity, a non-cardio activity, standing, sitting, walking, climbing stairs, descending stairs, hiking, jogging, sprinting, cycling, a treadmill exercise, and driving.

The generating of the body of the user may include classifying the physical activity of the user as either a cardio activity model or a non-cardio activity model by analyzing the received sensor signal, the prediction information may include information about calories to be expended for the physical activity, the information predicted by performing regression analysis by using heart rate data if the physical activity is classified as the cardio activity model, and the prediction information may include information about calories to be expended for the physical activity, the information predicted with reference to a calorie chart that shows a relation between the physical activity and calorie expenditure if the activity is classified as the non-cardio activity model.

The receiving may include receiving sensor signals with respect to the body of the user from a plurality of sensor, and classifying the physical activity of the user as one of the plurality of predefined activity models by using a correlation between the received sensor signals.

The receiving may include receiving at least one signal obtained by using at least one wearable sensor selected from the group consisting of a pedometer, a gyroscope, an accelerometer, a heart-rate monitor (HRM), a weight scale, and a barometer.

The generating of the prediction information about the body of the user may include determining a prediction model for generating the prediction information by performing regression analysis based on the received sensor signal, and the prediction model may be seamlessly recalibrated based on at least one selected from the group consisting of a change in the profile information about the user and a change in endurance of the user.

The generating of the prediction information of the physical body of the user may include determining a prediction model for generating prediction information about endurance expected in a future of the user, by performing regression analysis based on the received sensor signal, the outputting of the health care information may include outputting a workout plan for reaching a target endurance level based on a current endurance level of the user and the prediction model, and the prediction model may be a numerical formula model including at least one variable, selected from the group consisting of workout intensity and workout duration and a coefficient determined by using least square estimation.

According to an aspect of another exemplary embodiment, a non-transitory computer-readable recording storage medium having stored thereon a computer program which, when executed by a computer, may perform the method.

### BRIEF DESCRIPTION OF THE DRAWINGS

Exemplary embodiments are illustrated in the accompanying drawings, throughout which like reference letters indicate corresponding parts in the various figures.

These and/or other aspects will become apparent and more readily appreciated from the following description of the exemplary embodiments, taken in conjunction with the accompanying drawings in which:

FIG. 1 is a block diagram that includes various components for estimating daily energy expenditure during different activities, according to an exemplary embodiment;

FIG. 2 is a schematic diagram of a method of health care, which is performed by an apparatus, according to an exemplary embodiment;

FIG. 3 is a structural map of an apparatus for receiving a sensor signal from a wearable apparatus and creating a prediction model for a body, according to an exemplary embodiment;

FIG. 4 illustrates a correlation between received sensor signals according to an exemplary embodiment;

FIG. 5 illustrates physical activity recognition based on various physical activities, according to an exemplary embodiment;

FIG. 6A illustrates level classification into 3 levels for recognizing a physical activity, the recognizing being performed by the apparatus, according to an exemplary embodiment;

FIG. 6B illustrates level classification into 3 levels for recognizing a physical activity based on a received sensor signal, the recognizing being performed by the apparatus, according to an exemplary embodiment;

FIG. 7 illustrates various steps for creating a personalized prediction model so as to predict a trend in weight change management of a user, the creating being performed by the apparatus, according to an exemplary embodiment;

FIG. 8 illustrates creating of a prediction model for a weight change, which is performed by the apparatus, according to an exemplary embodiment;

FIG. 9 illustrates an example of a scenario for outputting health care information to a user about calorie intake based on calorie tracking analysis, which is performed by the apparatus, according to an exemplary embodiment;

FIG. 10 illustrates an example of a scenario for outputting health care information to a user based on food intake analysis, which is performed by the apparatus, according to an exemplary embodiment;

FIG. 11 illustrates a user interface of the apparatus according to an exemplary embodiment;

FIG. 12 illustrates modeling and visualizing of endurance estimated based on various cardio activities of an individual and providing of prediction information about endurance to a user, according to an exemplary embodiment;

FIG. 13A illustrates an apparatus for health care of a user, according to an exemplary embodiment;

FIG. 13B illustrates a flowchart of a method of health care of a user, according to an exemplary embodiment;

FIG. 14 illustrates determining of a prediction model that generates prediction information about future expected endurance of a user, which is performed by the apparatus, according to an exemplary embodiment;

FIG. 15 illustrates providing of various health care information relating to endurance of a user, which is performed by the apparatus, according to an exemplary embodiment;

FIG. 16A illustrates a user interface that may set a target endurance level of a user, which is performed by the apparatus, according to an exemplary embodiment;

FIG. 16B illustrates outputting of health care information variously according to an endurance level of a user, which is performed by the apparatus, according to an exemplary embodiment;

FIG. 17 illustrates a schematic diagram of a method of generating and providing a personalized fitness plan to an individual, which is performed by the apparatus, according to an exemplary embodiment;

FIG. 18 is a flowchart of generating of health care information that includes a fitness plan for a user, which is performed by the apparatus, according to an exemplary embodiment;

FIG. 19 illustrates a graph showing various heart rate zones and heart rate ranges associated with each heart range zone, according to an exemplary embodiment; and

FIGS. 20A through 20D illustrate screen shots of an application ‘fitness tracker’ for providing health care information to a user based on a created prediction model, according to an exemplary embodiment.

### DETAILED DESCRIPTION

Reference will now be made in detail to exemplary embodiments, examples of which are illustrated in the accompanying drawings, wherein like reference numerals refer to like elements throughout. In this regard, the present exemplary embodiments may have different forms and should not be construed as being limited to the descriptions set forth herein. Accordingly, the exemplary embodiments are merely described below, by referring to the figures, to explain aspects. As used herein, the term “and/or” includes any and all combinations of one or more of the associated listed items. Expressions such as “at least one of,” when preceding a list of elements, modify the entire list of elements and do not modify the individual elements of the list. Embodiments are provided so that this disclosure will be thorough and complete, and will fully convey the concept of the inventive concept to those skilled in the art, and the scope of embodiments of the inventive concept should be defined by the appended claims. General and widely-used terms have been employed herein, in consideration of functions provided in the inventive concept, and may vary according to an intention of one of ordinary skill in the art, a precedent, or emergence of new technologies. Additionally, in some cases, an applicant may arbitrarily select specific terms. Then, the applicant will provide the meaning of the terms in the description of the inventive concept. Accordingly, It will be understood that the terms, used herein, should be interpreted as having a meaning that is consistent with their meaning in the context of the relevant art and will not be interpreted in an idealized or overly formal sense unless expressly so defined herein. Hereinafter, example embodiments will be described in detail with reference to the accompanying drawings. It should be understood, however, that there is no intent to limit example embodiments to the particular forms disclosed, but on the contrary, example embodiments are to cover all modifications, equivalents, and alternatives falling within the scope of the inventive concept.

Additionally, a term ‘unit’ or ‘module’ means a hardware component or a circuit such as field programmable gate array (FPGA) or application-specific integrated circuit (ASIC).

Hereinafter, a term ‘personalize’ refers to optimization or specialization for a specific person, and a term ‘personalized prediction model’ refers to a model optimized or specialized for the specific person, and thus, may be applied only to the specific person. The personalized prediction model may refer to a model for generating prediction information useful for managing fitness of the specific person.

According to an exemplary embodiment, a method and apparatus for estimating daily energy expended during various physical activities or motions through accurate classification of physical activities and calorie estimation may be provided. Further, the method and apparatus may provide a personalized prediction model for predicting a trend in a weight change by managing the weight change. The method and apparatus may provide signal processing based on an approach, so as to identify and profile personalized physical activities by using sensor data and map expended calories with physical activities and motions corresponding to the expended calories.

The method and apparatus may provide accurate estimation of calories expended based on stratification of individual's day to day activities into a cardio or non-cardio activity model.

The method may accurately detect physical activities and motions that are classified as a cardio or non-cardio activity, and discover or estimate calories expended for each physical activity. Further, the method and apparatus may provide a personalized model based on accurate estimation of calories that are expended during a physical activity performed for a certain period of time. The personalized model may be used to predict weight change management.

Physical activity recognition is performed using sensor based analysis of body mechanics involving knowledge extraction from signal from sensors (which includes information extraction from signal emitted by a sensor). An intensity level of a physical activity may be determined based on stratification of heart rate zones against expended energy and the model may be personalized for a specific individual as it uses the individual's personal data. The method and apparatus may be used to track a fitness level of an individual. The tracking of the fitness level includes calorie intake tracking, physical activity recognition based on body mechanics, calorie expenditure tracking, prediction of calorie expenditure that may lead to weight loss, and fitness/wellness modeling.

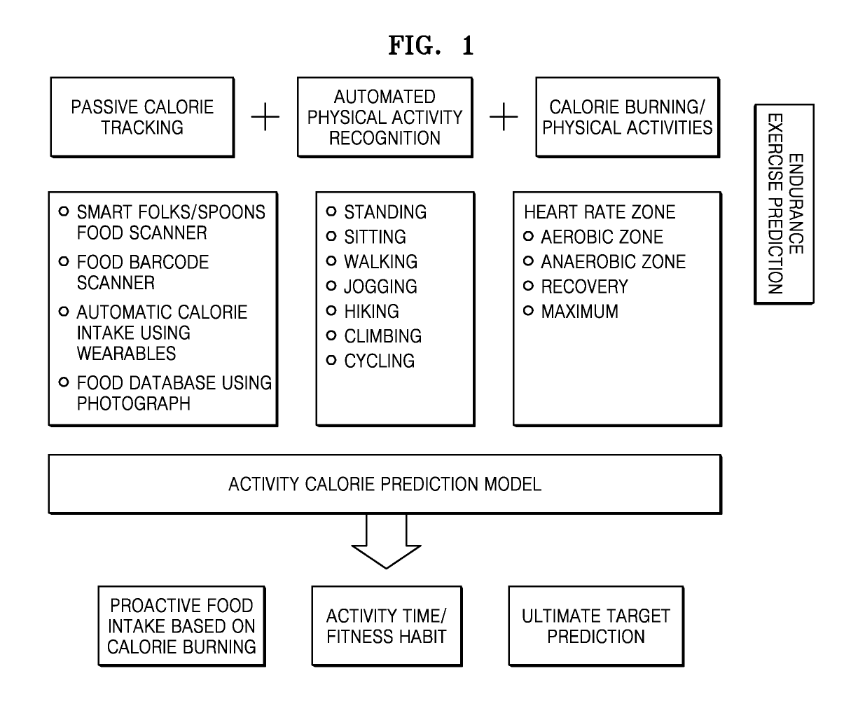

FIG. 1 is a block diagram that includes various components for estimating daily energy expenditure during different activities, according to an exemplary embodiment.

Various components shown in the block diagram include passive calorie tracking, automated physical activity recognition, and calorie burning/physical activities. Passive calorie tracking may include determination of a resting metabolic rate (RMR) or a basal metabolic rate. An activity calorie prediction model may generate proactive food intake information based on burnt calories, physical activity time and fitness habits, and prediction of a final goal.

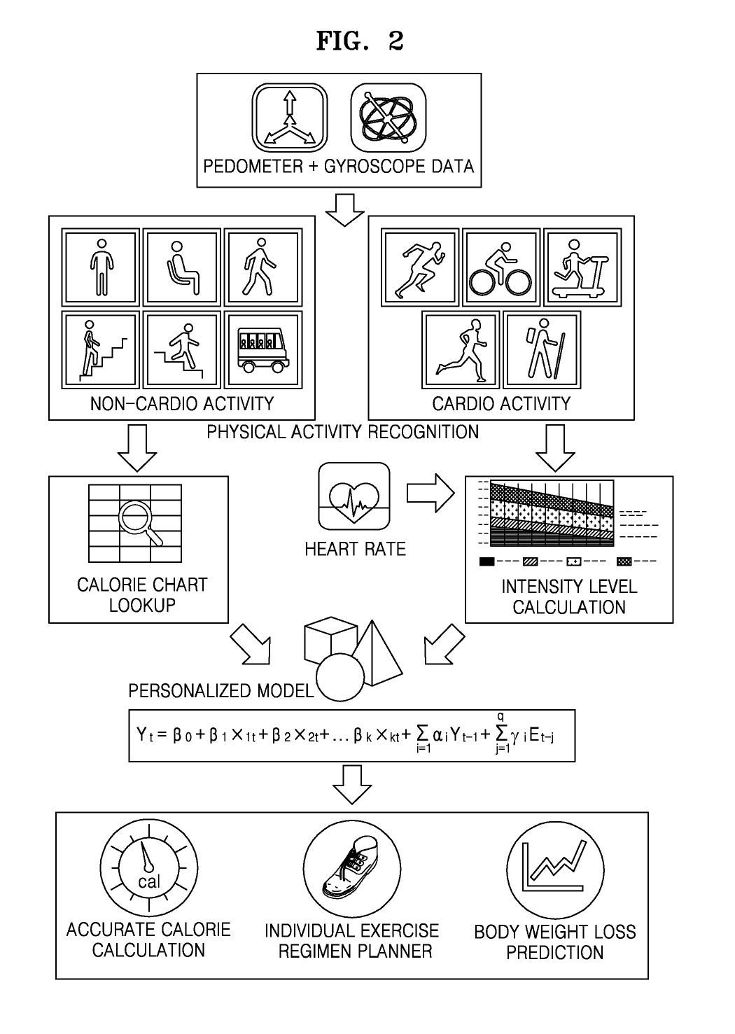

FIG. 2 is a schematic diagram of a method of health care, which is performed by an apparatus, according to an exemplary embodiment.

According to an exemplary embodiment, the apparatus for health care may obtain data via sensors such as a pedometer, an accelerometer, or a gyroscope, so as to classify physical activities of a user into cardio or non-cardio activities.

Calories expended by cardio activities may be estimated by using heart rate data (heart rate data may be obtained from a heart rate monitor) and an equation for associating the heart rate data with expended calories. Calories expended by non-cardio activities may be estimated by using calorie charts.

According to an exemplary embodiment, the method of health care may mathematically model data such as physical activities performed for a certain period of time and calories expended by an individual, so as to establish a fitness plan, predict a weight change, and accurately calculate calories.

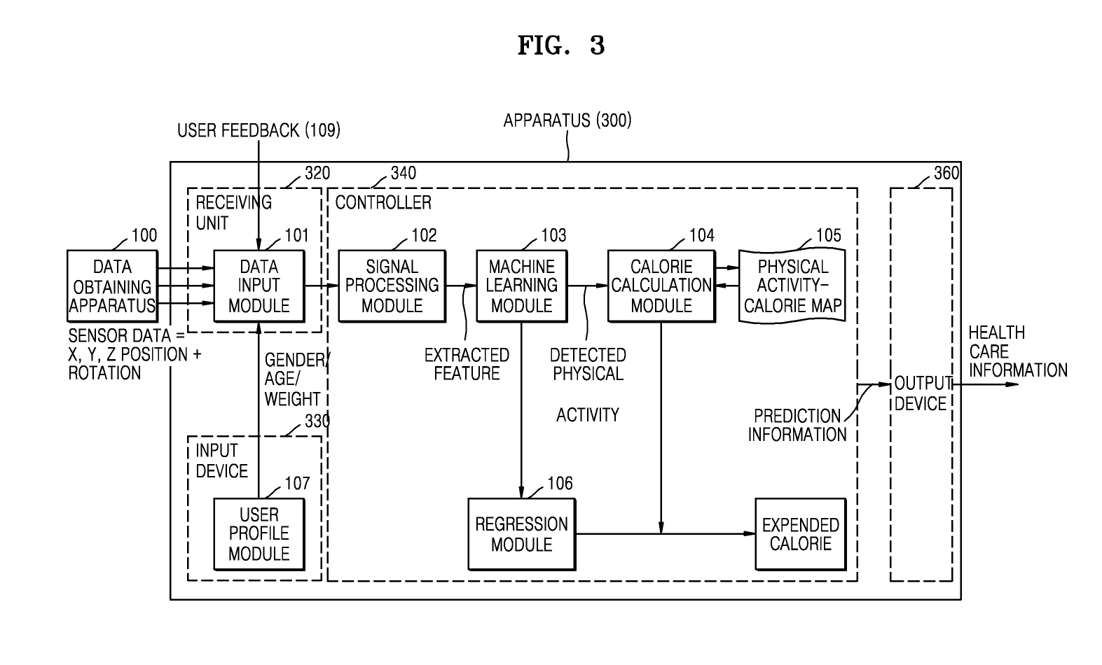

FIG. 3 is a structural map of an apparatus 300 for receiving a sensor signal from a wearable apparatus and creating prediction information about a physical body, according to an exemplary embodiment.

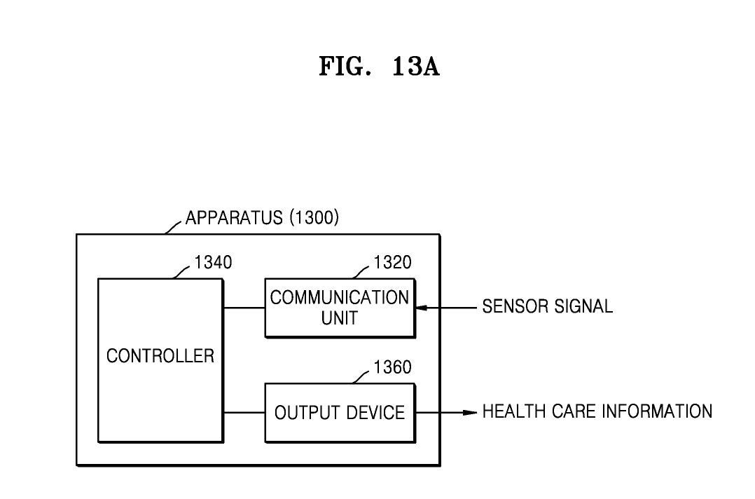

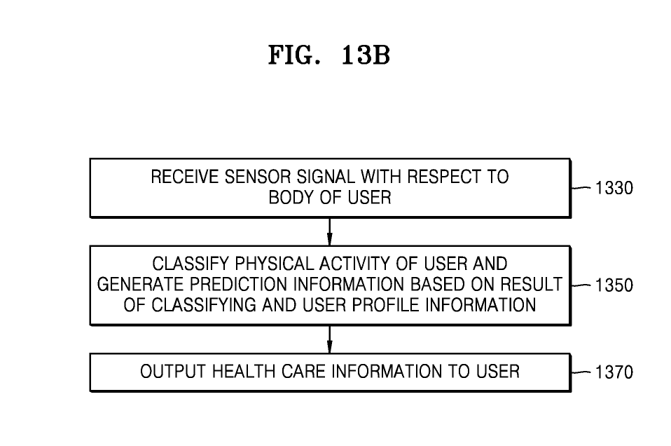

The apparatus 300 may include a receiving unit 320, a controller 340, and an output device 360. The apparatus 300 shows another exemplary embodiment of an apparatus 1300 shown in FIG. 13A. A communication unit 1320, a controller 1340, and an output device 1360 included in the apparatus 1300 may be respectively implemented as the receiving unit 320, the controller 340, and the output device 360, but are not limited thereto.

A data obtaining apparatus 100 may include a wearable apparatus. According to an exemplary embodiment, the data obtaining apparatus 100 may include at least one selected from the group consisting of a pedometer, a gyroscope, an accelerometer, a heart-rate monitor (HRM), a weight scale, a barometer, a magnetometer, a thermometer, a hygrometer, and an illuminometer sensor. The data obtaining apparatus 100 may be a sensor hub that is present in a mobile computing device (for example, a smartphone) or a wearable device. In FIG. 3, the data obtaining apparatus 100 is shown as being located outside the apparatus 300. However, the data obtaining apparatus 100 may be present inside the apparatus 300, or may include a plurality of wearable sensor that are be physically separate from each other.

The receiving unit 320 may receive a sensor signal (that is, data) regarding a physical body of a user from the data obtaining apparatus 100. The receiving unit 320 may be a circuitry or hardware component which receives a sensor signal. According to an exemplary embodiment, the receiving unit 320 may include a data input module 101. The data input module 101 may collect data received from the data obtaining apparatus 100. The data input module 101 may include a user feedback module 109 that allows intervention by a user on incorrect input data that is automatically selected by a computing device. The receiving unit 320 may receive from the input device 330 user profile information that includes information about at least one selected from the group consisting of a gender, an age, a height, a weight, and a body mass index (BMI).

The input device 330 may receive from a user an input of user profile information that includes information about at least one selected from the group consisting of a gender, an age, a height, a weight, and a BMI. The input device 330 may include a user profile module 107. The user profile module 107 includes database that contains data such as a gender, an age, a height, a weight, or a BMI. Information about a user, provided by the user profile module 107, may be used by the controller 340 to create a prediction model. In other words, the prediction model may be determined variously based on user profile information. [Equation 1] shows a formula for estimating calories to be expended while a same workout is performed according to user profile information, according to an exemplary embodiment. As shown in [Equation 1], even when a same physical activity is performed, calories to be expended by a user may vary depending on a gender, a weight, and an age of the user.

Male: ((−55.0969+(0.6309×HR)+(0.6309×HR)+(0.1988×W)+(0.2017×A))/4.184)×60×T

Female: ((−20.4022+(0.4472×HR)−(0.1263×W)+(0.074×A))/4.184)×60×T [Equation 1]

In [Equation 1], HR may represent a heart rate (in beats/min.), W may represent a weight (in kilograms), A may represent an age (year), and T may represent workout duration.

The controller 340 may classify a physical activity of a user as one of a plurality of predefined activity models, based on a sensor signal received by the receiving unit 320, and create prediction information about a physical body of a user based on a result of the classifying and user profile information. The prediction information about a physical body is useful information that may help health care of a user. The controller 340 may perform various regression analysis based on the received sensor signal and determine a prediction model, and then, create prediction information by using the determined prediction model. The controller 340 may be a processor, an application specific integrated circuit (ASIC), an embedded processor, a microprocessor, a hardware control logic, a hardware finite state machine (FSM), a digital signal processor (DSP), or a combination thereof.

The predefined activity model may include at least one selected from the group consisting of a cardio activity, a non-cardio activity, standing, sitting, walking, climbing/descending stairs, hiking, jogging, sprinting, cycling, a treadmill exercise, driving, a mild activity, a moderate activity, and a vigorous activity. The controller 340 may create prediction information about calories to be expended to perform a physical activity, according to the activity model obtained as a result of the classifying.

According to an exemplary embodiment, the controller 340 may process and analyze a signal obtained by the receiving unit 320, determine a physical activity of a user as either a cardio activity model or a non-cardio activity model, and then, differently estimate calories to be expended to perform the physical activity according to the determined type.

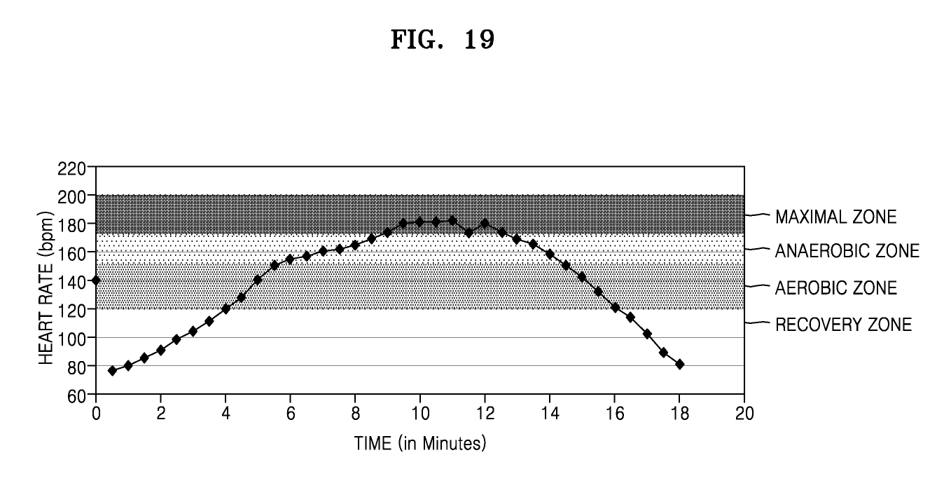

According to an exemplary embodiment, the controller 340 identify an intensity of a physical activity of the user based on a heart rate of the user, and determine the physical activity of the user as either a cardio activity model or a non-cardio activity model, based on the intensity of the physical activity of the user. Heart rates of the user may be classified into a plurality of heart rate zones that include an aerobic zone, an anaerobic zone, a recovery zone, and a maximal zone. A range of heart rates for each zone may be identified based on a maximum heart rate (MHR) of the user which may be obtained from an age of the user provided by user profile information. A mathematical modeling for an MHR and heart rate zones is described with reference to FIG. 19. If a physical activity is classified into a cardio activity model, the controller 340 may create prediction information that includes prediction information about calories to be expended for a physical activity by performing regression analysis by using heart rate data. If a physical activity is classified into a non-cardio activity model, the controller 340 may create prediction information about calories to be expended for a physical activity with reference to a calorie chart that shows a relation between a physical activity and calorie expenditure.

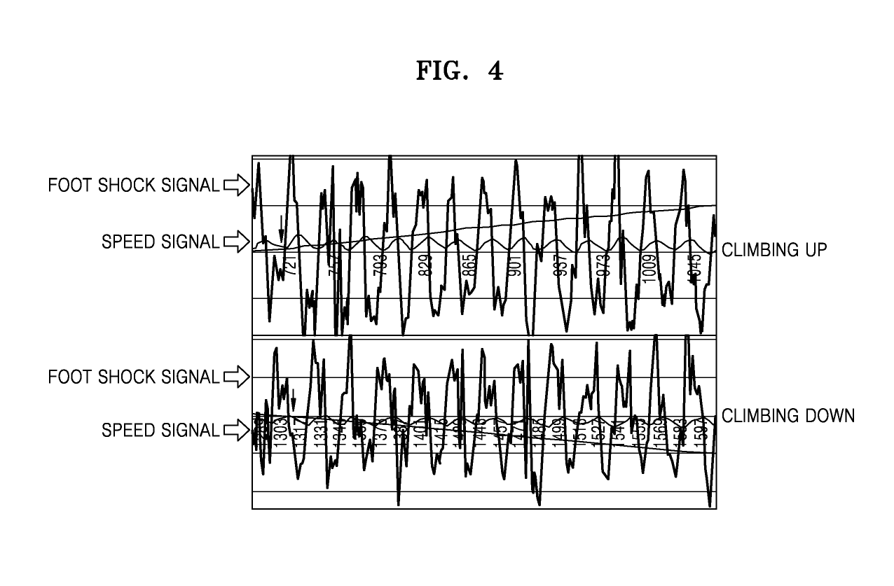

According to an exemplary embodiment, the controller 340 may classify physical activities of a user by using characteristics of a correlation between a plurality of signals with respect to a physical body of the user which are received by the receiving unit 320 from a plurality of sensor. An embodiment of classifying a physical activity of a user as one of a plurality of predefined activity models by using a correlation between a speed signal and a food shock signal is described with reference to FIG. 4.

According to an exemplary embodiment, the controller 340 can include a signal processing module 102, a machine learning module 103, a calorie calculation module 104, and a physical activity-calorie mapping module 105, and a regression module 106.

The signal processing module 102 may process and analyze a received sensor signal. A sensor signal may be received from an accelerometer, a gyroscope, a barometer, or a magnetometer. Signal processing may be performed on a client, a server, or both of them.

The machine learning module 103 may classify physical activities as various activity models. For example, the machine learning module 103 may classify user physical activities as one of resting, cardio, and non-cardio activities. If a type of a physical activity is determined as a cardio activity, the apparatus 300 may estimate calories to be expended while a non-cardio physical activity is performed, by using a statistical fitness model showing a relation between a heart rate and calorie expenditure. The statistical fitness model may be created by using information about at least one selected form the group consisting of a gender, an age, a height, a weight, and a BMI which are obtained by the receiving unit 320.

The calorie calculation module 104 may calculate calories with respect to a cardio activity model, by using a heart rate equation. According to an exemplary embodiment, the calculated calories may be used for the regression module 106 to perform regression analysis. In other words, information about the calculated calories may be used for various regression analysis so as to determine various prediction models for creating other prediction information. The apparatus 300 may perform accurate regression analysis by classifying physical activities of a user and estimating calories to be expended according to a result of the classifying.

The physical activity-calorie mapping module 105 may include database for a resting or non-cardio physical activity.

The regression module 106 may determine various prediction models for creating various prediction information for health care of a user, by performing various regression analysis. For example, the regression module 106 may perform regression analysis based on a particular physical activity and heart rate data which have been recorded for a certain period of time.

According to an exemplary embodiment, the regression module 106 may perform regression analysis by taking into account that energy expended while a treadmill exercise is taken is determined by intensity of the treadmill exercise, an inclination of a treadmill, a workout duration, and a weight of a user. The regression module 106 may determine a prediction model for creating prediction information about calories expended while a treadmill exercise is taken. A prediction module for predicting calories to be expended while a treadmill exercise is performed may be determined by using [Equation 2].

Cal(expended)=α₀+α₁Z₁+α₂Z₂+α₃Z₃+α₄Z₄+α₅1+f(W)Z [Equation 2]

where Cal may represent calories burnt during the exercise, Z₁ may represent a duration of time in a heart rate/speed zone—1, Z₂ may represent a duration of time in a heart rate/speed zone—2, Z₃ may represent a duration of time in a heart rate/speed zone—3, Z₄ may represent a duration of time in a heart rate/speed zone—4, f may represent a function with respect to a weight W, and I may represent an inclination of the treadmill. Accordingly, the controller 340 may classify a physical activity currently performed by the user as a treadmill exercise from among cardio physical activities, and estimate calories to be expended by the user while the user is taking treadmill exercise, by using the prediction model shown in [Equation 2].

According to another exemplary embodiment, the regression module 106 may determine a prediction model for generating prediction information about a future weight. The prediction model for predicting a future weight may be determined by using [Equation 3].

Yₜ=β₀+β₁X₁₁+β₂X₂ₜ+ . . . +βₖXₖₜ+ΣαᵢYₜ₋ᵢ+ΣYⱼεₜ₋ⱼ [Equation 3]

According to an exemplary embodiment, the regression module 106 may perform regression analysis for predicting a weight at a particular point of time by using auto regressive integrated moving average (ARIMA) modeling. In [Equation 3], Yₜ may represent a weight on day t, X₁ may represent an initial weight W₀, X₂ may represent energy intake, X₃ may represent energy expended by taking exercise (a HRM sensor and a pedometer), X₄ may represent energy expended by performing an average daily activity (pedometer data), and X₅ may represent a body fat rate. The regression module 106 may determine coefficients β₀, β₁ . . . βₖ, and αᵢ(I=1, 2, . . . p), and γⱼ(j=1, 2, . . . q) by substituting data that was collected for last several weeks into [Equation 3]. Even if X₁ to X₅ are unknown, the regression module 106 may predict a future weight by using the determined coefficients.

According to another embodiment, the regression module 106 may determine a prediction model for generating prediction information about endurance of a user. An example of a prediction model for generating information about endurance of a user may be determined by using [Equation 4].

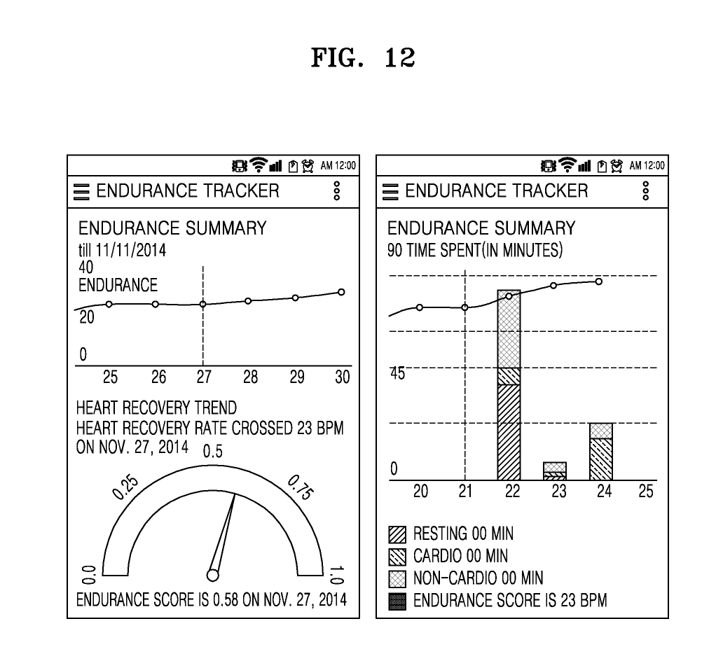

Endurance=β₀y₀+Σβₖyₖ(0=<k<=n) [Equation 4]

where y₀ may represent initial endurance of a user, yₜ may represent intensity of a current workout, y₂ may represent a type of the workout, y₃ may represent an age of the user, y₄ may represent a level of previous training, y₅ may represent a weight, y₆ may represent a lifestyle habit, and y₇ may represent a heart rate. The prediction model for generating prediction information about endurance of a user will be described later in detail with reference to FIGS. 12 and 14 through 20D.

The controller 340 may seamlessly change a prediction model adaptively based on at least one selected from the group consisting of a change in user profile information and a change in endurance of a user. The controller 340 may construct a prediction model for an individual person based on previous record data, compare data that is newly collected thereafter to the previous record data included in the constructed prediction model, and thus, check whether the newly collected data matches or is similar to the previous record data. If the newly collected data is greatly different from the previous record data in the prediction model, health or fitness of the person may be determined as being improved or worsened, a notification or warning message may be transmitted to the person. The person may periodically recalibrate a statistical prediction model. Since comparison of data with sample data based on a model may be quickly performed in real time and a space of the sample data is small, the comparison may be performed by a client apparatus without intervention by a server, and easily maintained.

According to an exemplary embodiment, the output device 360 may output health care information to a user based on prediction information. The output device 360 may include a liquid crystal display (LCD), a thin-film transistor-liquid crystal display (TFT-LCD), an organic light-emitting diode (OLED), a flexible display, a 3-dimensional (3D) display, and an electrophoretic display, but is not limited thereto.

Health care information may include prediction information or include information that is derived from the prediction information, such as warning or notification to a user. For example, the apparatus 300 may output a sentence for warning or suggesting calorie intake to a user, based on prediction information about calorie intake of a user for a day and prediction information about calories expended by the user for a day. Health care information may include at least one selected from the group consisting of a number of steps that a user has taken for a day, a distance for which the user has moved for a day, an amount of expended calories, endurance, an recommended amount of food intake, an amount of necessary calorie intake, nutrients or ingredients of consumed food, and a weight change, but is not limited thereto. Additionally, health care information may include a workout plan or a fitness plan for a user to achieve target endurance. The output device 360 may visualize or numeralize, and then, provide health care information to the user.

FIG. 4 illustrates a correlation between received sensor signals according to an exemplary embodiment.

FIG. 4 illustrates physical activity recognition of the subject based on various signals captured by one or more sensors of an electronic device, according to the embodiments as disclosed herein. The proposed method utilizes different types of sensors. For example, instantaneous signal sensed by the gyrometer provides features for climbing stairs and the accelerometer provides shock data, where the accelerometer and the gyrometer are situated in the same electronic device that is held in the same location/position of the body (such as hand or pocket) of the subject. The proposed method utilizes a phase correlation between the output signals from the accelerometer and the gyrometer. The phase correlation is highly accurate in classifying the physical activity. For example, climbing up and climbing down signals shown in FIG. 4 correspond to opposite correlation of the zero crossing time of the foot shock with the zero velocity time. Thus, the proposed method provides kind of sensor signal correlation based study of body or bio-mechanics and extracting the information about the physical activity being performed from the correlation.

Another example providing sensor signal correlation for identifying or recognizing the physical activity is described below. While it is known that magnetometer provides stable directional data, the proposed method utilizes this data to precisely characterize the drift in the constant coefficients and integrating the distance from the accelerometer. This helps in identifying drastic change in direction such as turning.

In another example, shape of the accelerometer signal as captured in various transforms (as Fourier boundary descriptor) for each physical activity improves the accuracy of classification of activities that are very close in feature space. Further, the instantaneous signal from the accelerometer corresponding to a stride pattern of the subject and foot impact of the subject are analyzed to identify optimal personal design of walking outfits for the subject and impact of terrains on the stride pattern.

Accordingly, the machine learning module 103 may determine a physical activity of a user as stair-climbing motions or stair-descending motions by using a foot shock signal and a speed signal processed by the signal processing module 102.

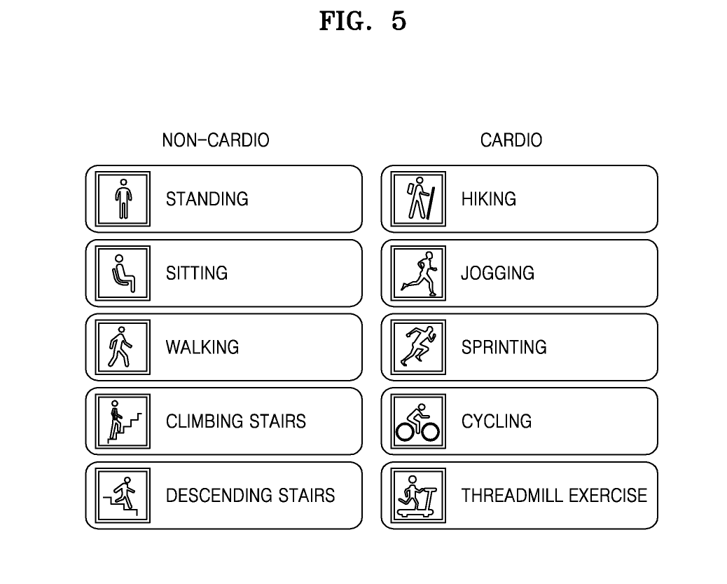

FIG. 5 illustrates physical activity recognition based on various physical activities of a user, according to an exemplary embodiment.

According to an exemplary embodiment, the apparatus 300 may classify physical activities of the user as a non-cardio activity or a cardio activity. According to an exemplary embodiment, daily physical activities performed by the user may automatically classified by using an accelerometer, a gyrometer, and a magnetometer. According to an exemplary embodiment, the apparatus 300 may classify a physical activity of the user as a non-cardio activity, and then, subdivide the physical activity to be specified as standing, sitting, walking, stair climbing, stair descending, or the like.

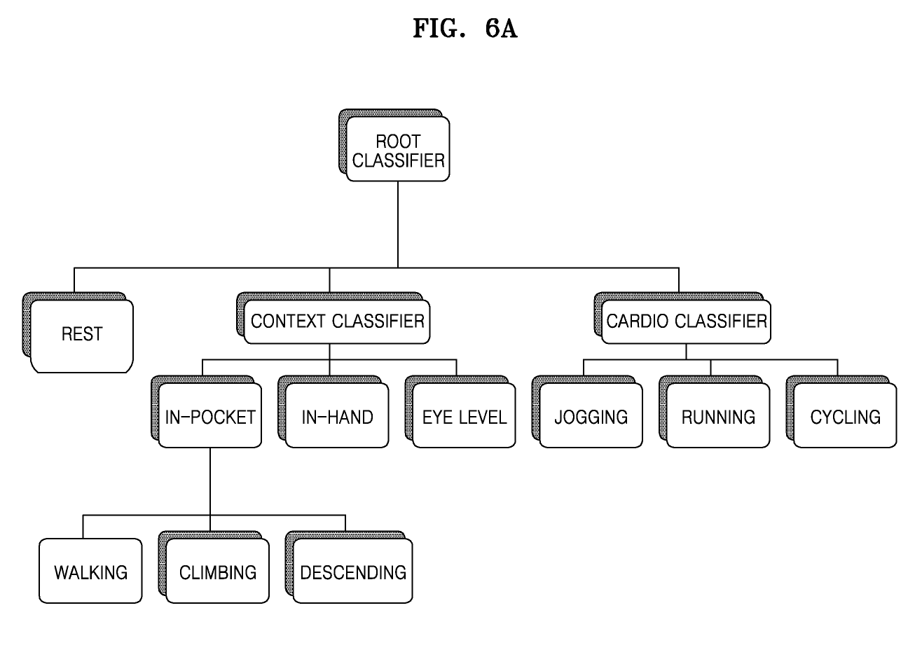

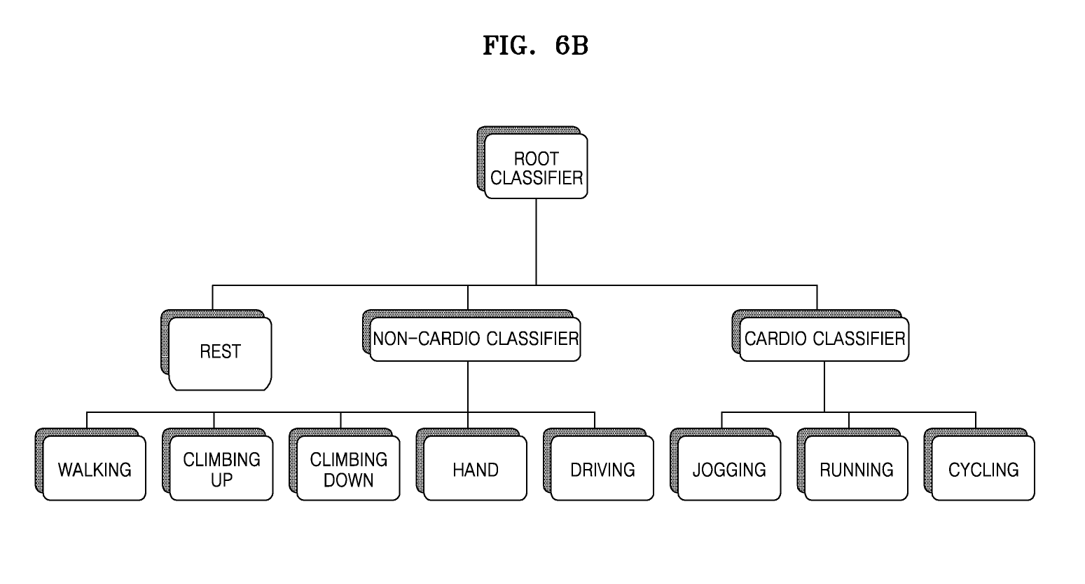

FIG. 6A illustrates level classification for recognizing a physical activity, the recognizing being performed by the apparatus, according to an exemplary embodiment.

For example, a context classifier may determine 3 available modes of possessing a mobile device (that is, in a pocket, in a hand, or before eyes), with respect to a non-cardio physical activity, and may assume that the mobile device is placed in a trouser pocket of the user with respect to a cardio activity.

FIG. 6B illustrates level classification into 3 levels for recognizing a physical activity based on a received sensor signal, the recognizing being performed by the apparatus, according to an exemplary embodiment.

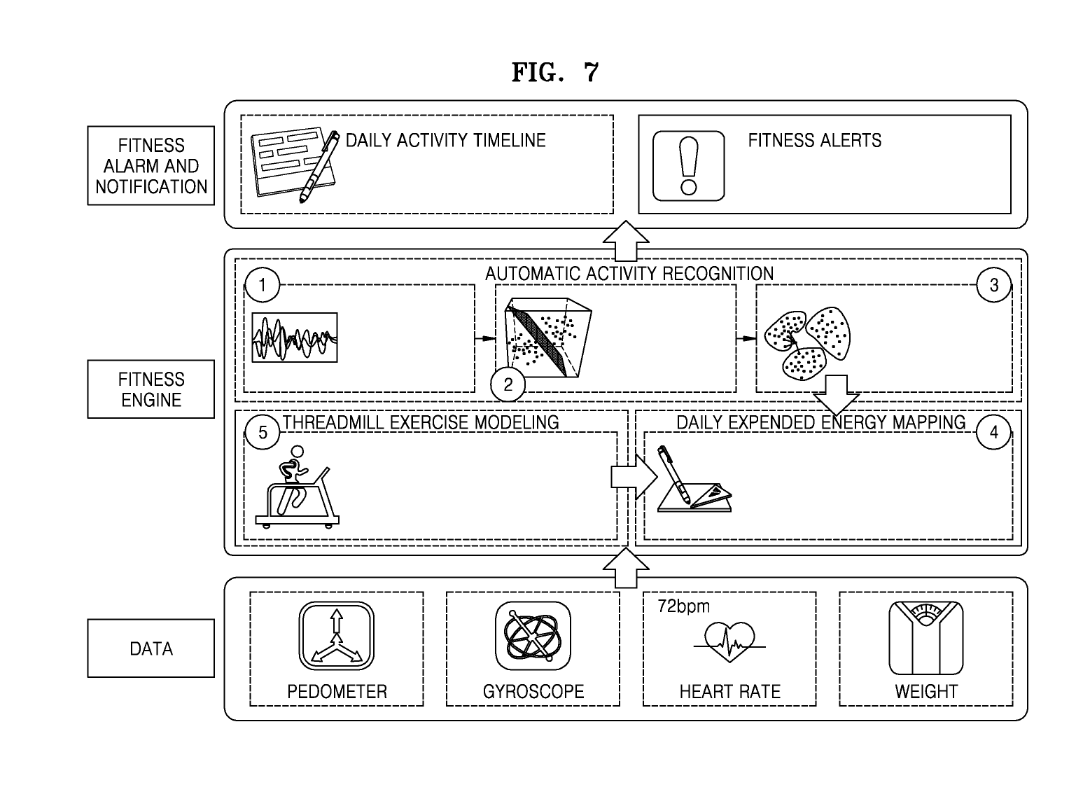

FIG. 7 illustrates various steps for creating a personalized prediction model so as to predict a trend in weight change management of a user, the creating being performed by the apparatus, according to an exemplary embodiment.

A data obtaining step may include obtaining various data from one or more sensors such as a pedometer, a gyroscope, a HRM monitor, or a weight scale, but is not limited thereto.

A fitness engine step may include signal processing, machine learning, activity labeling, daily expended energy mapping, and treadmill exercise modeling.

The signal processing may include noise filtering and feature extraction. The machine learning may include classification of physical activities by using a random forest or an artificial neural network (ANN). The physical activity labeling may include selection of an algorithm having a highest accuracy, based on false-positive analysis. The treadmill exercise modeling may include calculation of calorie, burnt while treadmill exercise is taken, by using a statistical model. The daily expended energy mapping may include calorie mapping. The calorie mapping may be performed by looking up a calorie chart and a workout duration for recognizing a physical activity with respect to a non-cardio physical activity, and by using calories calculated from a treadmill exercise model with respect to a cardio physical activity.

A fitness alarm and notification step may include performing at least one inference from a fitness engine so as to provide useful health information. According to an exemplary embodiment, the alarm and notification step may include writing a daily activity timeline and alerting fitness to a user. The writing of the daily physical activity timeline may include accurate and automatic tracking of energy (in calories) expended for a day, by using a familiar user interface. The fitness alerting may include classifying a physical activity level of a user as a level ranged from a low-level activity such as sitting to a high-level activity, and provide a fitness alarm so that the user may take exercise or a fitness state of the user may be recognized.

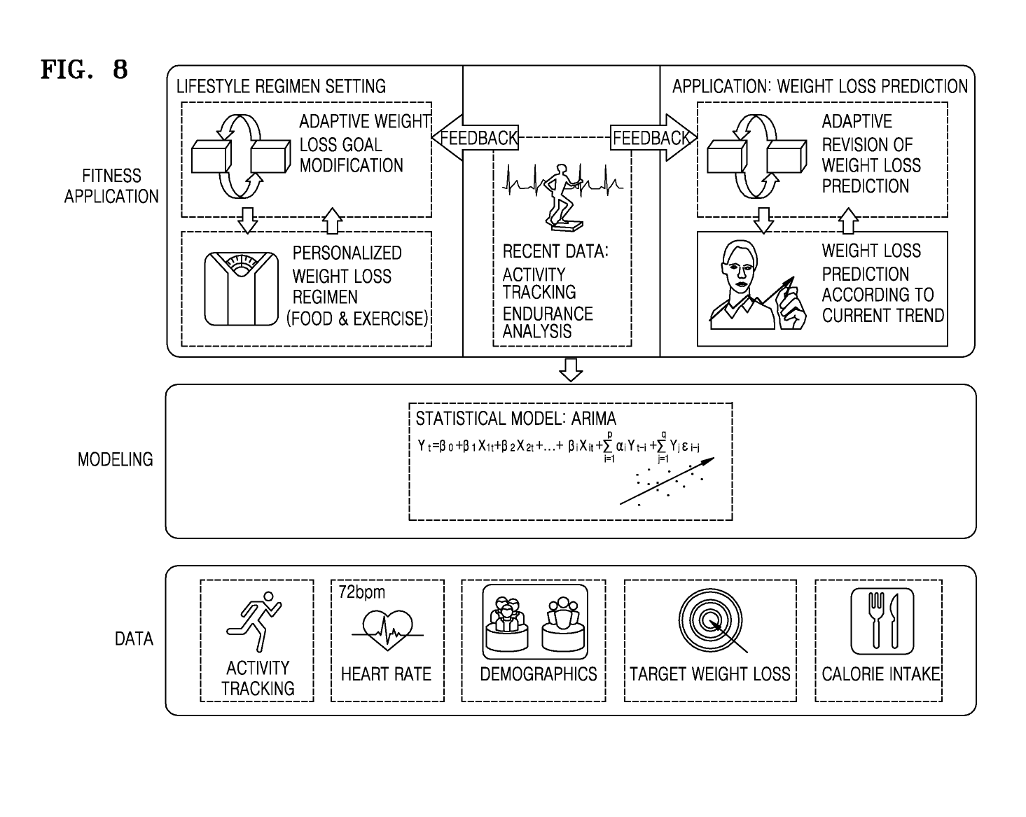

FIG. 8 illustrates creating of a weight change prediction model, which is performed by the apparatus, according to an exemplary embodiment.

The weight change prediction model may include data, modeling, and a fitness application. The data may include physical activity tracking obtained from a pedometer, a heart rate, a population statistics data, a target weight loss, or an amount of calorie intake.

Modelling may include modeling performed by using a statistical model.

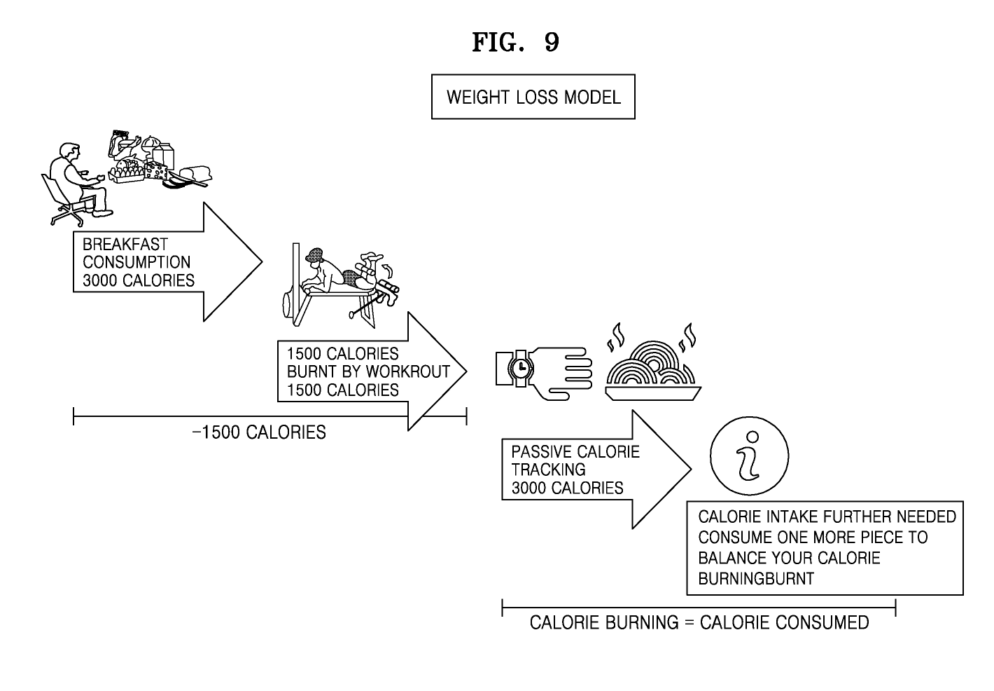

FIG. 9 illustrates an example of a scenario for outputting health care information to a user about calorie intake based on calorie tracking analysis, which is performed by the apparatus 300, according to an exemplary embodiment.

According to an exemplary embodiment, the apparatus 300 may track calories expended by a person for a day, and track and profile a physical activity. Passive calorie tracking analysis may provide a recommended amount of food for compensating for calorie burning and various information to a user in advance, based on calorie burning generated at a beginning of a day. Additionally, if expended calories exceed predetermined calories, the apparatus 300 may provide to a user information about an amount of calories which is further needed for a remaining period of time for a day.

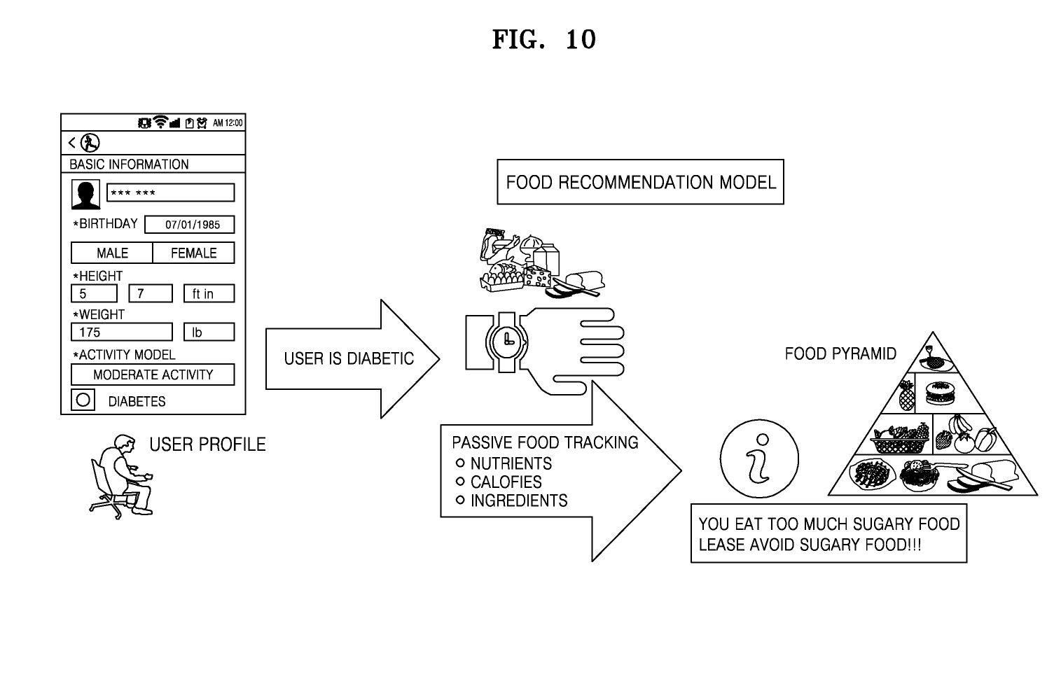

FIG. 10 illustrates an example of a scenario for outputting health care information to a user based on food intake analysis, which is performed by the apparatus 300, according to an exemplary embodiment.

The apparatus 300 may perform food intake analysis for detecting nutrient, ingredient, and calorie intake by a user. Various methods of estimating calories, for example, by using food scanners, food database, automatic calorie intake tracking wearable devices or the like, may be employed for food intake analysis. For example, if consumed food contains too much sugar/carbohydrate, the apparatus 300 may warn this to the user. The apparatus 300 may determine a food recommending model based on a food pyramid and a user profile.

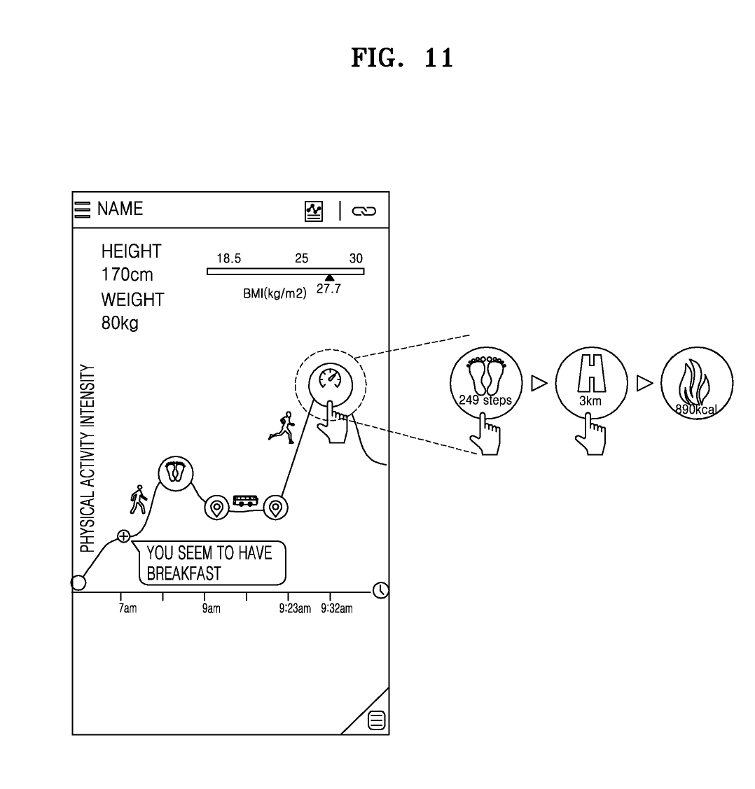

FIG. 11 illustrates a user interface of the apparatus 300 according to an exemplary embodiment. The apparatus 300 may show a profile of a user for a day as a number of steps that the user has taken, a distance for which the user has moved, and an amount of expended calories for the day.

FIG. 12 illustrates modeling and visualizing of endurance estimated based on various cardio activities of a user and providing of prediction information about endurance to the user, according to an exemplary embodiment.

Endurance is one of important indices of a physical level of a person. Endurance is defined as an ability of taking exercise under a certain load for a long period of time, or taking exercise under an increased load for a same period of time. Medically, heart rate recovery may be a good index for indicating endurance. For example, endurance may be defined based on a difference between a peak heart rate and a heart rate that is measured 1 minute after exercise is stopped. In other words, endurance may be indicated by a degree of how fast a peak heart rate is dropped to the heart rate that is measured 1 minute after exercise is stopped. That is, the apparatus 300 may determine current endurance by using heart rate recovery data obtained from exercise taken by an individual. The heart rate recovery data may be determined by using [Equation 5].

HRR₁ₘᵢₙ=HRₚₑₐₖ−HR₁ₘᵢₙ [Equation 5]

where HRₚₑₐₖ refers to a peak heart rate, and HRᵢₘᵢₙ refers to a heart rate obtained 1 minute after the exercise is stopped.

Heart Rate Recovery (HRR) during the first minute−HRR₍₁ₘᵢₙ₎ after exercise is due to parasympathetic reactivation. Extent of parasympathetic reactivation is an important indicator of cardiorespiratory fitness which is defined as Endurance. the HRR data is utilized quantitatively monitor fitness over a period of time. The HRR data provides a comprehensive approach for predicting endurance covering relevant exercise parameters derived from an objective model. Further, the created endurance model can be employed to design a personalized workout plan for a target fitness or time. The endurance capacity estimation through HRR₁ₘᵢₙ approach is not adopted currently by any of the existing mobile application.

FIG. 12 illustrates windows that showing endurance trend and contribution of proportion of each physical activity model (resting, cardio, and non-cardio activities) to endurance, which are provided by an endurance tracker. Endurance trend may provide health care information that includes summary information about endurance of a user, by plotting a change in endurance scores of the user for a certain period of time such as a change in endurance scores for past days and prediction of an endurance score obtained based on recently observed fitness of the user.

FIG. 13A illustrates an apparatus 1300 for health care of a user, according to an exemplary embodiment.

The apparatus 1300 may include the communication unit 1320, the controller 1340, and the output device 1360. The apparatus 1300 may be implemented as the apparatus 300 shown in FIG. 3, and the communication unit 1320, the controller 1340, and the output device 1360 included in the apparatus 1300 may be respectively implemented as the receiving unit 320, the controller 340, and the output device 360 included in the apparatus 300, but are not limited thereto. Accordingly, descriptions that were provided with regard to the apparatus 300 may also be applied to the apparatus 1300, even if the descriptions thereof are not provided here again.

The communication unit 1320 may receive a sensor signal with respect to a physical body of a user from a wearable apparatus. According to an exemplary embodiment, the communication unit 1320 may receive a sensor signal with respect to the physical body from one or more wearable sensors that are present inside or outside the apparatus 1300. The wearable sensor may include at least one selected from the group consisting of a pedometer, a gyroscope, an accelerometer, a HRM monitor, a weight scale, a barometer, a magnetometer, a thermometer, a hygrometer, and an illuminometer sensor. The communication unit 1320 may receive from an input device (not shown) user profile information that includes information about at least one selected from the group consisting of a gender, an age, a height, a weight, and a BMI. The communication unit 1320 is a hardware circuit that enables communication with outside by using a communication route. For example, the communication route may include a route of a wireless communication, wired communication, optics, ultrasonic waves, or a combination thereof. Satellite communication, mobile communication, Bluetooth, an infrared data association standard (IrDA), wirelessfidelity (WiFi), and worldwide interoperability for microwave access (WiMAX) are examples of wireless communication that may be included in the communication route. Ethernet, a digital subscriber line (DSL), fiber to the home (FTTH), and a plain old telephone service (POTS) are examples of wired communication that may be included in the communication route. Additionally, the communication route may include a personal area network (PAN), a local area network (LAN), a metropolitan area network (MAN), a wide area network (WAN), or a combination thereof.

The controller 1340 may classify a physical activity of a user as one of a plurality of predefined activity models, based on a sensor signal received by the receiving unit 1320, and generate prediction information about a physical body of the user based on a result of the classifying and user profile information. The prediction information is useful information that may help health care of a user, and may include a weight, an amount of expended or consumed calories, endurance, or a number of steps that the user has taken. The predefined activity model may include at least one selected from the group consisting of a cardio activity, a non-cardio activity, standing, sitting, walking, climbing/descending stairs, hiking, jogging, sprinting, cycling, a treadmill exercise, driving, a mild activity, a moderate activity, and a vigorous activity. The controller 1340 may employ a machine learning method or use characteristics of a correlation between a plurality of signals with respect to the physical body of the user received from a plurality of sensors, so as to classify a physical activity of a user as one of the plurality of predefined activity models. The controller 1340 may determine various prediction models for generating various prediction information by performing regression analysis based on the received sensor signal. The prediction information is useful information that may help health care of a user, and may create prediction information such as endurance, calories, a weight, or a nutrition state. The controller 1340 may variously predict calories to be expended according to classification of a physical activity of a user. The controller 1340 may determine various prediction models for generating other prediction information, by performing regression analysis by using the predicted calories. The prediction model may be created variously based on at least one selected from the group consisting of a gender, an age, a height, a weight, and a BMI. The controller 1340 may be a processor, an ASIC, an embedded processor, a microprocessor, a hardware control logic, an FSM, a DSP, or a combination thereof.

The output device 1360 may output health care information to a user based on the prediction information. The output device 1360 may include a LCD, a TFT-LCD, an OLED, a flexible display, a 3D display, or an electrophoretic display, but is not limited thereto. Health care information may include prediction information or derived information that is based on the prediction information, such as warning or notification to a user. For example, the apparatus 1300 may output a sentence warning or suggesting calorie intake to a user, based on prediction information about calories consumed by a user for a day and prediction information about calories expended by the user for a day. Health care information may include at least one selected from the group consisting of a number of steps that a user has taken for a day, a distance for which the user has moved for a day, an amount of expended calories, endurance, an recommended amount of food intake, an amount of necessary calorie intake, nutrients or ingredients of consumed food, and a weight change, but is not limited thereto. The output device 1360 may visualize or numeralize, and then, provide health care information to the user.

FIG. 13B illustrates a flowchart of a method of health care of a user, which is performed by the apparatus 1300, according to an exemplary embodiment.

Since FIG. 13B shows a flowchart of a method of health care of a person, which is performed by the apparatus 1300 shown in FIG. 13A, descriptions that were provided with regard to the apparatus 1300 may also be applied to the method described with reference to FIG. 13B, even if the descriptions are not provided here again.

In operation 1330, the apparatus 1300 may receive a sensor signal with respect to a physical body of a user from a wearable apparatus. According to an exemplary embodiment, the apparatus 1300 may receive a sensor signal (that is, sensor data) indicating a physical activity of the user from one or more sensors from among a pedometer, a gyroscope, an accelerometer, a HRM monitor, a weight scale, a barometer, a magnetometer, a thermometer, a hygrometer, and an illuminometer sensor. The one or more sensors may be located in a mobile device or a wearable device. In operation 1330, the apparatus 1330 may further receive user profile information such as a gender, an age, a height, a weight, or a BMI.

In operation 1350, the apparatus 1300 may classify a physical activity of a user as one of a plurality of predefined activity models, based on a sensor signal received by the receiving unit 320, and generate prediction information about the physical body of the user based on a result of the classifying and user profile information. In operation 1350, the apparatus 1300 may perform various regression analysis based on the received sensor signal, determine a prediction model, and then, generate prediction information by using the determined prediction model. The prediction information refers to useful information that may help health care of a user, and a prediction model is a personalized model for generating the prediction information. For example, the apparatus 1300 may process and analyze a signal obtained in operation 1330, and thus, classify a physical activity of the user as one of a cardio activity model and a non-cardio activity model, and then, differently estimate calories expended to perform a physical activity according to a result of the classifying. Alternately, the apparatus 1300 may classify a physical activity of a user by using characteristics of a correlation between a plurality of signals with respect to a physical body of the user which are received by the receiving unit 320 from a plurality of sensor. The apparatus 1300 may estimate calories by using an equation showing a relation between a physical activity and expended calories with respect to a cardio activity, and estimate calories expended while a physical activity is performed by using a calorie map with respect to a non-cardio activity. For example, the apparatus 1300 may determine a prediction model obtained by using regression analysis, so as to estimate calories to be expended during a treadmill exercise session that is classified as a cardio activity. Additionally, the apparatus 1300 may estimate calories expended during various physical activities, and determine a prediction model for estimating a future weight by substituting the estimated calories in a regression equation. An equation showing a relation between a regression model or a physical activity between expended calories may be constructed by using user profile information received in operation 1330. Additionally, the prediction model may be seamlessly recalibrated so that endurance or a weight change of a person is reflected in the prediction model.

In operation 1370, the apparatus 1300 may show health care information to a user. The health care information may include at least one selected from the group consisting of a number of steps that a user has taken, a distance for which the user has moved, an amount of expended calories, endurance, an recommended amount of food intake, an amount of necessary calorie intake, nutrients or ingredients of consumed food, and a weight change, but is not limited thereto. The apparatus 1300 may visualize or numeralize, and then, provide health care information to the user.

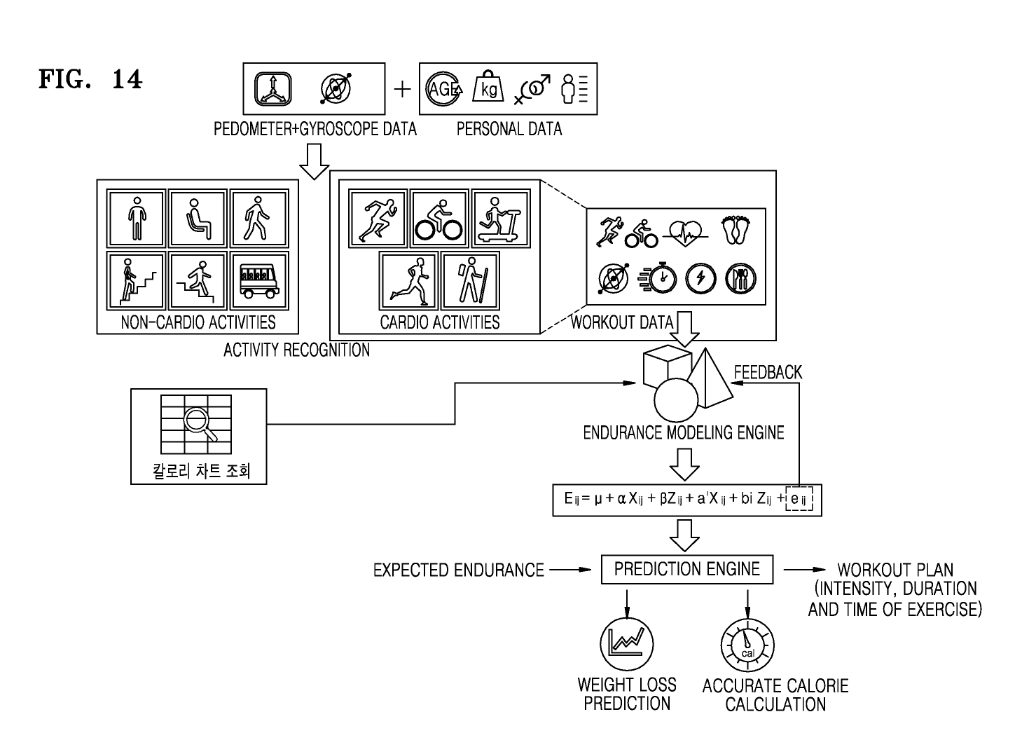

FIG. 14 illustrates determining of a prediction model that generates prediction information about future expected endurance of a user, which is performed by the apparatus 300, according to an exemplary embodiment.

The apparatus 300 may seamlessly observe and update an endurance level of a user, by accurately calculating a current endurance level of the user by using heart rate data such as heart rate recovery data and observing daily physical activities of the user. The heart rate recovery data may be obtained by using the method described with reference to FIG. 12. The apparatus 300 may provide appropriate health care information to the user based on the calculated endurance level.

The apparatus 300 may accurately classify a physical activity of the user by using a sensor signal received from a wearable apparatus, calculate calories expended on the classified physical activity based on user profile information and physical activity data, and periodically predict an endurance level of the user. The apparatus 300 may create a prediction model for generating prediction information about future expected endurance of the user (hereinafter, referred to as an endurance prediction model), by using time series statistical modeling based on data on physical activities of the user collected for a certain period of time. The endurance prediction model may be a numerical formula model that includes a coefficient determined by using at least one variable selected from the group consisting of a workout intensity and a workout duration and least square estimation. The controller 340 included in the apparatus 300 may estimate current endurance of the user by using heart rate recovery data received by the receiving unit 320, and determine a workout plan necessary for reaching a target endurance level by using the endurance prediction model, and then, the output device 360 may output the determined workout plan to the user.

The controller 340 may determine an endurance prediction model based on a wearable sensor signal received by the receiving unit 320. The endurance prediction model may be a longitudinal model with workout intensity and session duration (or time) as two multilevel factors. The endurance model may be created dynamically according to a fitness state of the user for a certain period of time. According to an exemplary embodiment, the apparatus 300 may model intra-subject dependence based on a correlated error. The apparatus 300 may model endurance by using least square estimation with respect to a model parameter. An endurance level of a user may vary depending on at least one selected from an intensity level, session time, intensity and a session, and time interaction. The apparatus 300 create an endurance prediction model by using [Equation 6], but creating of an endurance prediction model is not limited thereto. The apparatus 300 may determine an endurance prediction model, by performing regression analysis by using various time series variables for determining endurance of a user. The endurance prediction model may include at least one variable selected from the group consisting of an intensity level and a workout duration. Each coefficient may be determined by using the least square estimation.

Eᵢⱼ=μ+αXᵢⱼ+βZᵢⱼ+ai′Xᵢⱼ+biZᵢⱼ+eᵢⱼ [Equation 6]

where Eᵢⱼ refers to expected endurance, (Xᵢⱼ, Zᵢⱼ) refers to a design point specific to a workout plan and subject-specific covariates, (μ, α, β) are fixed effect coefficients, and (ai, bi) are subject-specific random coefficients, and the errors eᵢⱼ are such that cov(eᵢⱼ, e_{i′j′})=0 if i is not equal to i′ and else it is nonzero.

According to an exemplary embodiment, the apparatus 300 may determine an optimal workout plan for achieving a target endurance level based on a current endurance level and a target endurance level, by using the endurance prediction model shown in [Equation 6]. The apparatus 300 may determine an optimum combination of factors such as workout intensity or time for a specific subject group so as to reach the target endurance level. The apparatus 300 may compare effects of endurance changes for respective factors with each other by fixing other factors. The apparatus 300 may optimize a model for factors for a specific session time. The apparatus 300 may incorporate different exercise factors to compare multiple endurance curves to each other. The apparatus 300 may determine an optimum workout plan to reach the target endurance level as quickly as possible in a fixed time span. In other words, the apparatus 300 may optimize an endurance model with respect to various factors so as to get a steepest ascent endurance curve to accomplish the target endurance level.

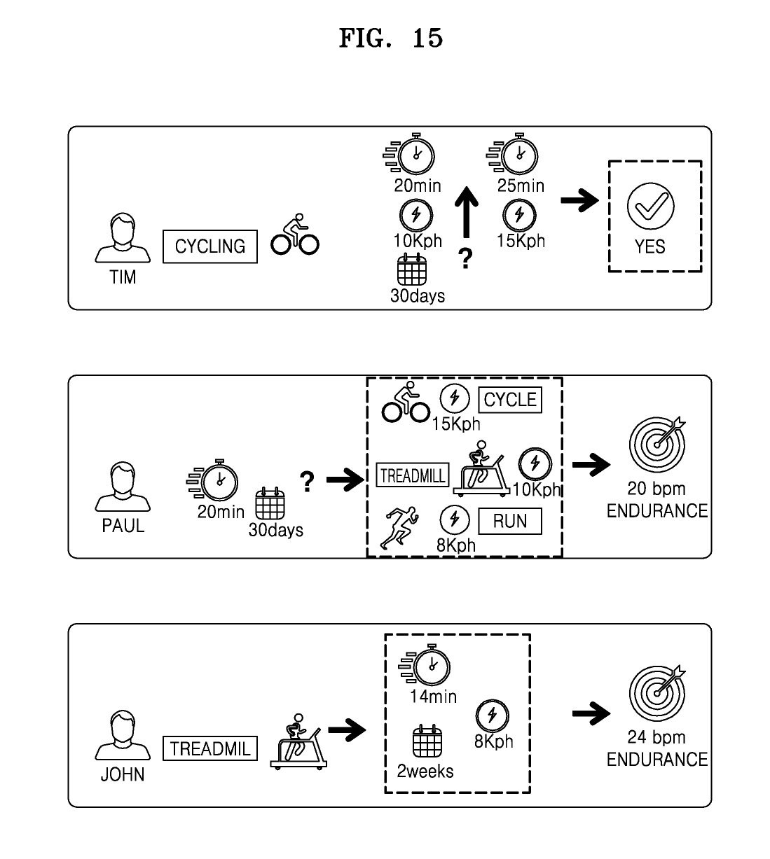

FIG. 15 illustrates providing of various health care information relating to endurance of a user, which is performed by the apparatus 300, according to an exemplary embodiment.

The apparatus 300 may output a workout plan for the user to take exercise within a fixed time span so as to reach a target endurance level.

According to an exemplary embodiment, if the user has cycled at 10 kph for 20 minutes for 30 days, the apparatus 300 may output to the user information that the user may comfortably cycle at 15 kph for 25 minutes based on an endurance level of the user.

According to another exemplary embodiment, if a user has worked out for 20 minutes everyday for one month, the apparatus 300 may provide to the user an effective workout plan for improving endurance by 20 bpm.

According to another exemplary embodiment, if a user wants to achieve target endurance of 24 bpm, the apparatus 300 may provide information about intensity, duration and session time of a treadmill exercise necessary for achieving the target endurance to the user

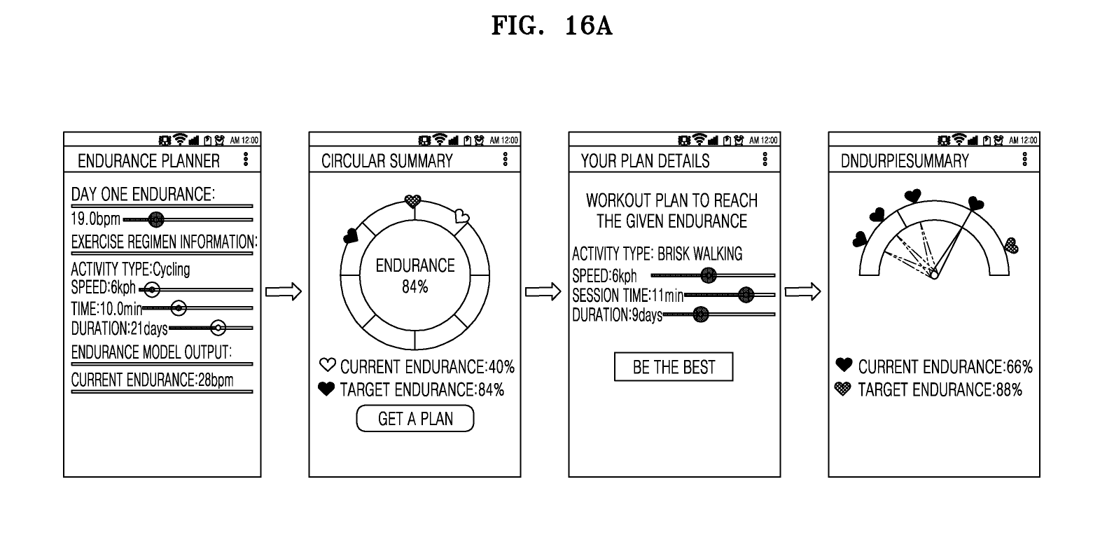

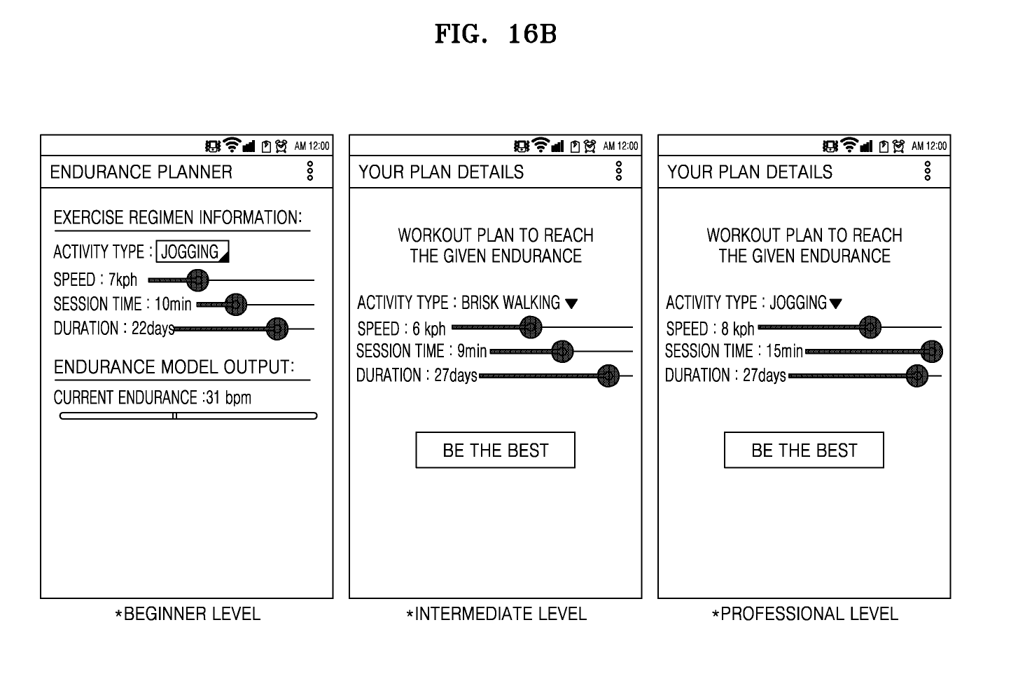

FIG. 16A illustrates a user interface for setting set a target endurance level of a user, which is performed by the apparatus 300, according to an exemplary embodiment. The user interface shown in FIG. 16A may be displayed on the output device 360.

The apparatus 300 may indicate an initial endurance level. The apparatus 300 may categorize a current endurance level of the user as a beginner, an intermediate, or a professional, assess an endurance level that may be attained by the user according to a result of the classifying, and allow the user to reach a target endurance level, and measure a current endurance level of the user so as to track a relative progress with respect to the current endurance level. The apparatus 300 may provide exercise optimized and personalized parameters so that the user may achieve the target endurance level. An endurance map may be provided to the user so that the user may track a progress with respect to the target endurance level set by the user. The endurance map may provide a snapshot of various endurance stages that have been reached by the user for a certain period of time.

FIG. 16B illustrates outputting of health care information variously according to an endurance level of a user, which is performed by the apparatus 300, according to an exemplary embodiment.

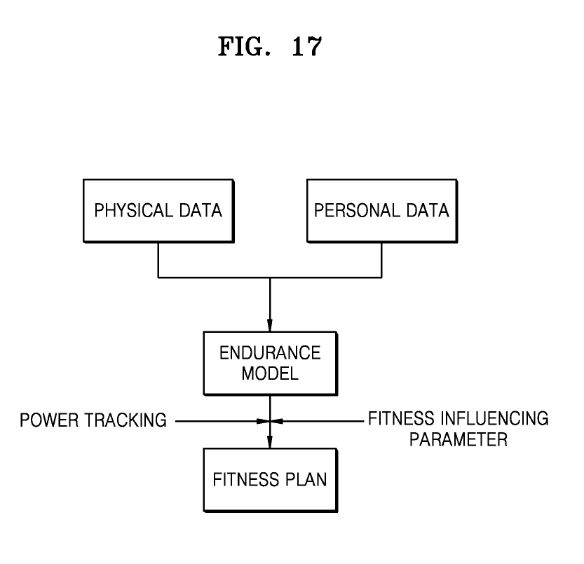

FIG. 17 illustrates a schematic diagram of a method of generating and providing a personalized fitness plan to an individual, which is performed by the apparatus 300, according to an exemplary embodiment.

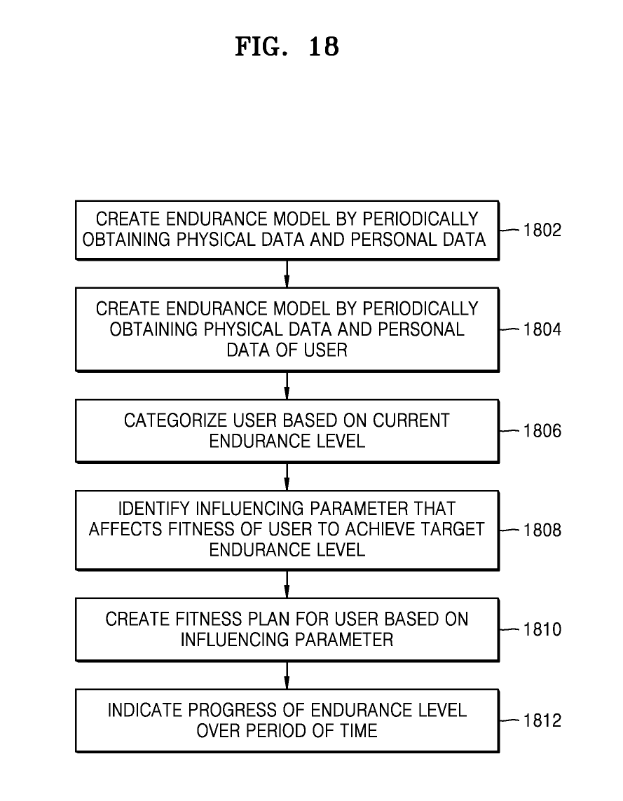

The method, described with reference to FIGS. 17 and 18, may be performed by the controller 340 included in the apparatus 300 or the controller 1340 included in the apparatus 1300, but performing of the method is not limited thereto.

The apparatus 300 may create a prediction model for generating prediction information about endurance (that is, an endurance model) based on calorie expenditure or intake. Additionally, the user's target endurance level may be determined from the endurance model, and parameters for improving fitness and achieving the target endurance level may be suggested. The parameters may include, for example, workout intensity, a workout duration, or lifestyle habits. According to an exemplary embodiment, the workout plan may be set by taking muscular fitness into account based on power tracking. For example, power tracking may be performed by using a resting heart rate, heart rate recovery, or maximal oxygen consumption (VO2max). Power training, suggested to the user, may increase muscle mass, improve a metabolic rate, improve/maintain bone density, improve overall strength and fitness, reduce blood lipids, and improve a functional capability.

Physical data may include workout data and heart rate data of the user. The workout data may include any type of data with respect to physical activities of the user. Personal data may include user profile information that includes at least one selected from the group consisting of a gender, an age, a height, a weight, a BMI.

FIG. 18 is a flowchart of generating of health care information that includes a fitness plan for a user, which is performed by the apparatus 300, according to an exemplary embodiment. The flowchart shown in FIG. 18 may be performed by a microcontroller, a microprocessor, the controller 340, or a computer-readable recording storage medium.

In operation 1802, the apparatus 300 may create an endurance model by periodically obtaining physical data and personal data of a user. The endurance model is fine-tuned to a good extent, and thus help to choose contributions from subtle factors, such as, impact on endurance if the workouts are done in-doors or out-doors, impact on endurance due to terrain on which workouts are done, impact of endurance from particular food habits followed by user.

In operation 1806, the apparatus 300 may categorize the user as a beginner, an intermediate, or a professional based on current endurance. The apparatus 300 may categorize the user based on the present endurance as a beginner, an intermediate, or a professional, assess the user's best possible endurance according to a result of the categorizing, allow the user to set a goal, and measure present endurance of the user so as to track a relative progress with respect to the current endurance. The apparatus 300 may provide exercise parameters that are optimized and personalized so that the user achieves the target endurance level. According to an exemplary embodiment, the apparatus 300 may assess the user's best possible endurance according to a result of the categorizing, and identify influencing parameters that affect fitness of the user. The influencing parameters may include, for example, workout intensity, a workout duration, lifestyle habits or the like. In operation 1810, the apparatus 300 may create health care information that includes a fitness plan for the user, based on the influencing parameters.

According to an exemplary embodiment, the apparatus 300 may take power tracking into account in creating the fitness plan for the user. Whereas endurance training improves sustained energy utilization for a long period of time, power training is important for building muscular fitness that leads to instant energy release for consumption in short bursts. Power tracking and the endurance model together may form an integral component in creating an overall fitness plan for the user.

The apparatus 300 may enable the user's fitness equipment and an electronic device to communicate with a cloud and store preferences setting and workouts for a specific user. The apparatus 300 may help the user to set a goal, participate in challenges, and socialize with their fitness community on a website and mobile application.

FIG. 19 illustrates a graph indicating various heart rate zones and associated heart rate range for each heart rate zone, according to an exemplary embodiment. The line graph shown in FIG. 19 is a real time plot of variation of the heart rate of the subject through various heart rate zones when the subject is performing a cardio physical activity involving higher intensity exercise for a specific time span observed. The heart rate zones are determined based on the MHR of the subject that is obtained from the profile data, where MHR=208−0.7×age.

For the example considered in the FIG. 19, the range of each heart rate zone computed based on the MHR, along with consumption of fat and carbohydrates for each heart rate zone is given below:
Recovery zone: <65% of MHR; fat 85%, carbohydrates 15%
Aerobic zone: 65%-75% of MHR; fat 60%, carbohydrates 40%
Anaerobic zone: 75%-95% of MHR; fat 20%, carbohydrates 80%
Maximal zone: 90%-100% of MHR; fat 10%, carbohydrates 90%

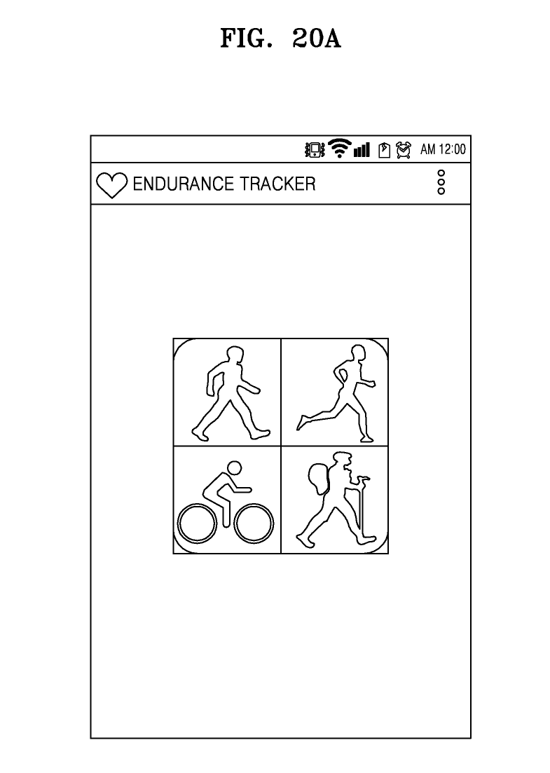

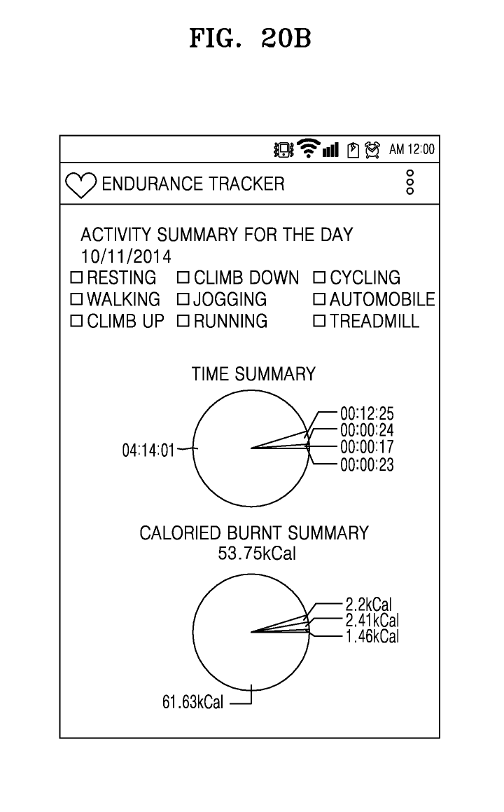

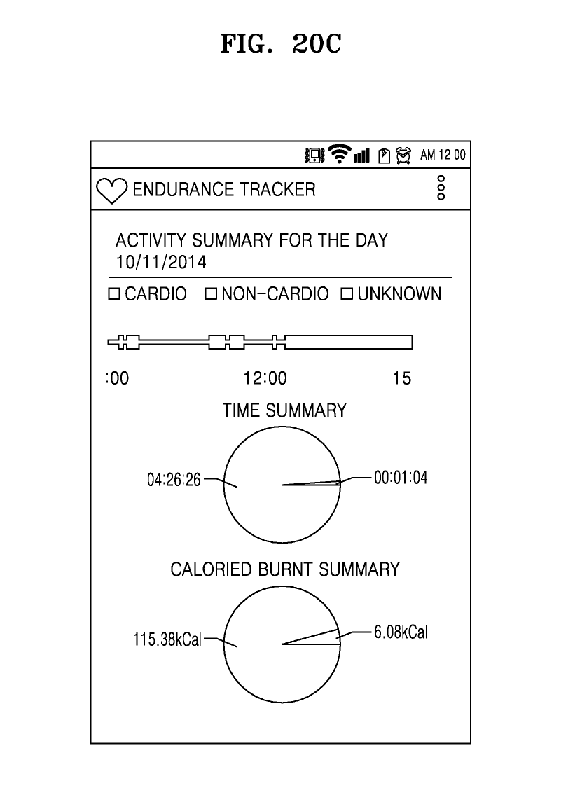

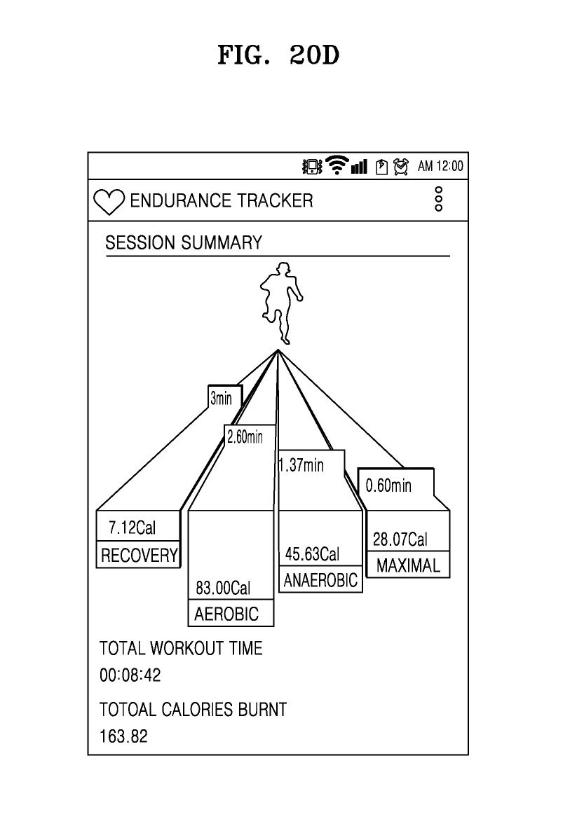

FIGS. 20A through 20D illustrate screen shots of an application ‘fitness tracker’ for providing health care information to a user based on a created prediction model, according to an exemplary embodiment. The screen shots shown in FIGS. 20A through 20D may be screens shown by the output device 360 included in the apparatus 300.

FIG. 20B shows an activity summary window of the endurance tracker. The activity summary window may highlight various physical activities performed by the user throughout a day in pie charts that indicate a time span for each physical activity and calories burnt (calories expended) for each corresponding physical activity. For example, if the user performs jogging, running, climbing up, climbing down, resting, cycling, a treadmill exercise, all these activities, identified and discriminated, may be displayed on the window. Further, a time duration and calories expended during each physical activity may be respectively indicated in the pie charts.

FIG. 20C indicates an activity summary window of the endurance tracker in which cardio/non-cardio classification of the physical activities performed for the day is displayed. For example, if the subject walks, rests or jogs, the activity summary window highlights both cardio and non-cardio indicators on the display window. Further, the pie charts for calories consumed and a time duration of each physical activity performed may be also displayed in the activity summary window.

FIG. 20D shows a heart rate zone window providing a session summary for a particular exercise session (a cardio physical activity) performed by the user. The session summary may include a time duration for which a heart of the user was in a particular heart rate zone, total workout duration, and calories consumed during the workout. For example, during a treadmill exercise session for 8 minutes and 42 seconds, the user's heart rate may be recorded to have been in a recovery zone for 3 minutes, in an aerobic zone for 2.6 minutes, in an anaerobic zone for 1. 37 minute, and in a maximal zone for 0.6 minute. This information may indicate to the user whether the user have taken a particular exercise efficiently. Further, tracking of a third hear rate zone window for a certain time span may also indicate to the user whether workout performance of the user has been improved or degraded for the time span. Such indications may enable the user to take corrective measures if required to improve a health fitness. The apparatus 300 may provide automatic notification and health tips to improve fitness based on a current fitness model.

The method provided with reference to FIGS. 14 through 20D are described as being performed by the apparatus 300. However, the operations provided with reference to FIGS. 14 through 20D may also be performed by the apparatus 1300.

The above-described exemplary embodiment may also operate on at least one hardware device, and may be implemented through at least one software program for performing network management functions of controlling the above-described elements.

The method of health care may also be embodied as computer-readable codes on a computer-readable recording medium. The computer-readable recording medium is any data storage device that can store data which can be thereafter read by a computer system. Examples of the computer-readable recording medium include read-only memory (ROM), random-access memory (RAM), CD-ROMs, magnetic tapes, floppy disks, optical data storage devices, and carrier waves (such as data transmission through the Internet). The computer-readable recording medium can also be distributed over network coupled computer systems so that the computer-readable code is stored and executed in a distributed fashion.

In the inventive concept, a process, an apparatus, a product, and/or a device are simple, cost-effective, not complicated, greatly various, and accurate. Additionally, according to exemplary embodiments, well-known components are applied to the process, the apparatus, the product, and/or the device so that manufacture, applications, and utilization may be efficiently and economically implemented and immediately used. Additionally, the process, the apparatus, the product, and/or the device meet a current trend that require cost reduction, apparatus simplification, and performance improvement. According to an exemplary embodiment, this will resultantly at least enhance a level of current technology.

While this inventive concept has been particularly shown and described with reference to exemplary embodiments thereof, it will be understood by those skilled in the art that various deletions, substitutions, and changes in form and details of the apparatus and method, described above, may be made therein without departing from the spirit and scope of the inventive concept as defined by the appended claims. Therefore, the scope of the inventive concept is defined not by the detailed description of the inventive concept but by the appended claims, and all differences within the scope will be construed as being included in the inventive concept.

It should be understood that exemplary embodiments described herein should be considered in a descriptive sense only and not for purposes of limitation. Descriptions of features or aspects within each exemplary embodiment should typically be considered as available for other similar features or aspects in other exemplary embodiments.

While one or more exemplary embodiments have been described with reference to the figures, it will be understood by those of ordinary skill in the art that various changes in form and details may be made therein without departing from the spirit and scope as defined by the following claims.

What is claimed is:

1. An apparatus comprising:
a receiving unit for receiving a sensor signal for a body of a user from a wearable apparatus;
a controller for classifying a physical activity of the user as one of a plurality of predefined activity models based on the received sensor signal, and generating prediction information about the body of the user based on a result of the classifying and profile information about the user; and
an output device for outputting health care information to the user based on the prediction information.

2. The apparatus of claim 1, wherein the predefined activity models comprise at least one selected from the group consisting of a cardio activity, a non-cardio activity, standing, sitting, walking, climbing/descending stairs, hiking, jogging, sprinting, cycling, a treadmill exercise, and driving.

3. The apparatus of claim 1, wherein the prediction information comprises information about differently predicted calories to be expended to perform the physical activity of the body, according to the activity model obtained by the classifying.

4. The apparatus of claim 3, wherein the controller classifies the physical activity of the user as either a cardio activity model or a non-cardio activity model by analyzing the received sensor signal,
if the physical activity is classified as the cardio activity model, the prediction information comprises information about calories to be expended for the physical activity, the information predicted by performing regression analysis based on heart rate data, and
if the activity is classified as the non-cardio activity model, the prediction information comprises information about calories to be expended for the physical activity, the information predicted with reference to a calorie chart that shows a relation between the physical activity and calorie expenditure.

5. The apparatus of claim 1, wherein the receiving unit receives sensor signals with respect to the body of the user from a plurality of sensor, and
the controller classifies the physical activity of the user as one of the plurality of predefined activity models by using a correlation between the received sensor signals.

6. The apparatus of claim 1, wherein the controller determines a prediction model for generating prediction information about endurance expected in a future of the user, by performing regression analysis based on the received sensor signal.

7. The apparatus of claim 6, wherein the prediction information comprises information about calories to be expended to perform the physical activity, according to the activity model obtained by the classifying, and
the prediction model is a numerical formula model comprising at least one variable selected from the group consisting of workout intensity and workout duration and a coefficient determined by least square estimation based on the information about the calories.

8. The apparatus of claim 6, wherein the receiving unit receives heart rate data of the user from the wearable apparatus, and
the controller determines a current endurance level based on the received heart rate data and determines a workout plan for achieving a target endurance level based on the prediction model, and
the output device outputs to the user health care information that includes the determined workout plan.

9. The apparatus of claim 1, wherein the prediction information comprises information about a future body weight which is predicted by performing regression analysis by applying regressive integrated moving average (ARIMA) modeling to the received sensor signal.

10. The apparatus of claim 1, wherein the sensor signal is a signal obtained by using at least one wearable sensor selected from the group consisting of a pedometer, a gyroscope, an accelerometer, a heart-rate monitor (HRM), a weight scale, and a barometer.

11. The apparatus of claim 1, further comprising an input device for receiving an input of the profile information from the user,
wherein the profile information comprises information about at least one selected from the group consisting of a gender, an age, a height, a body weight, and a body mass index (BMI) of the user.

12. The apparatus of claim 1, wherein the controller determines a prediction model for generating the prediction information by performing regression analysis based on the received sensor signal, and
the prediction model is seamlessly recalibrated based on at least one selected from the group consisting of a change in the profile information about the user and a change in endurance of the user.

13. The apparatus of claim 1, wherein the output device displays to the user at least one selected from the group consisting of an amount of expended calories, endurance, an recommended amount of food intake, an amount of necessary calorie intake, nutrients or ingredients of consumed food, and a weight change.

14. An apparatus comprising a controller for creating an endurance model for predicting endurance of a user by obtaining physical data of the user and user profile information, identifies one or more parameters that affect fitness of the user, and creating a fitness plan for the user based on the identified parameters.

15. The apparatus of claim 14, wherein the physical data of the user comprises workout data and heart rate data of the user.

16. The apparatus of claim 14, wherein the controller measures the physical data of the user, measures calories consumed and calories expended by the user and creates a prediction model, generates an endurance score of the user, and generates health care information of the user for a certain period of time based on the prediction model.

17. A method comprising:
receiving a sensor signal for a body of a user from a wearable apparatus;
classifying a physical activity of the user as one of a plurality of predefined activity models based on the received sensor signal, and generating prediction information about the body of the user based on a result of the classifying and profile information about the user; and
outputting health care information to the user based on the prediction information.

18. The method of claim 17, wherein the predefined activity models comprise at least one selected from the group consisting of a cardio activity, a non-cardio activity, standing, sitting, walking, climbing stairs, descending stairs, hiking, jogging, sprinting, cycling, a treadmill exercise, and driving.

19. The method of claim 17,
wherein the generating of the prediction information of the physical body of the user comprises determining a prediction model for generating prediction information about endurance expected in a future of the user, by performing regression analysis based on the received sensor signal,
the outputting of the health care information comprises outputting a workout plan for reaching a target endurance level based on a current endurance level of the user and the prediction model, and
the prediction model is a numerical formula model comprising at least one variable, selected from the group consisting of workout intensity and workout duration, and a coefficient determined by using least square estimation.

20. A non-transitory computer-readable recording storage medium having stored thereon a computer program which, when executed by a computer, performs the method of claim 17.
